(19) United States
(12) Patent Application Publication
(10) Pub. No.: US 2022/0301716 A1
(43) Pub. Date: Sep. 22, 2022

(54) MEDICAL INFORMATION PROCESSING APPARATUS, MEDICAL INFORMATION LEARNING APPARATUS, MEDICAL INFORMATION DISPLAY APPARATUS, AND MEDICAL INFORMATION PROCESSING METHOD

(71) Applicant: Canon Medical Systems Corporation, Otawara-shi (JP)

(72) Inventors: Yusuke KANO, Nasushiobara (JP); Yudai YAMAZAKI, Nasushiobara (JP)

(73) Assignee: Canon Medical Systems Corporation, Otawara-shi (JP)

(21) Appl. No.: 17/695,618

(22) Filed: Mar. 15, 2022

(30) Foreign Application Priority Data
Mar. 19, 2021 (JP) 2021-046180
Mar. 7, 2022 (JP) 2022-034591

(51) Int. Cl. G16H 50/20; G16H 50/30; G16H 50/70; G16H 10/60

(52) U.S. Cl. CPC G16H 50/20; G16H 50/30; G16H 50/70; G16H 10/60

(57) ABSTRACT

A medical information processing apparatus include a processing circuitry. The processing circuitry obtains medical care information relating to medical care events of a target patient. The processing circuitry maps the medical care information on a first graph to generate a second graph relating to the target patient. The first graph includes nodes corresponding to the medical care events and edges indicative of a relationship between the nodes. The processing circuitry estimates medical judgment information relating to the target patient, based on the second graph relating to the target patient.

### CROSS-REFERENCE TO RELATED APPLICATIONS

This application is based upon and claims the benefit of priority from Japanese Patent Application No. 2021-046180, filed Mar. 19, 2021; and No. 2022-034591, filed Mar. 7, 2022; the entire contents of all of which are incorporated herein by reference.

### FIELD

Embodiments described herein relate generally to a medical information processing apparatus, a medical information learning apparatus, a medical information display apparatus, and a medical information processing method.

### BACKGROUND

By constructing a database by systematizing classifications of diseases and a relationship between diseases as medical knowledge, it can be expected to utilize the database for the understanding of complex diseases, the discovery of research hypotheses, medical care support, and the like. For example, in the field of medicine, the development of medical ontology, such as ICD-10 that is a classification system of diseases, or SNOMED-CT that describes semantic relations between medical terms, has been in progress. In addition, in recent years, attention has been paid to a data analysis method using a graph structure that can express a relationship between medical care events.

Fluctuation exists in diseases. In other words, one definite criterion does not always exist for a disease. Even in the case of the same disease, various states of the disease are present, the definition of the disease varies across the ages, and, in some cases, fluctuation occurs in judgment depending on doctors who diagnose the disease.

### BRIEF DESCRIPTION OF THE DRAWINGS

FIG. 1 is a view illustrating a configuration example of a medical information system according to an embodiment.

FIG. 2 is a conceptual view of a medical knowledge graph.

FIG. 3 is a view illustrating a configuration example of a medical information processing apparatus illustrated in FIG. 1.

FIG. 4 is a view illustrating a flow of a medical information process by the medical information processing apparatus illustrated in FIG. 3.

FIG. 5 is a view illustrating an example of a mapping process relating to step SA2 in FIG. 4.

FIG. 6 is a view illustrating an example of an estimation process relating to step SA3 in FIG. 4.

FIG. 7 is a view illustrating an example of a display screen that is displayed in step SA4 in FIG. 4.

FIG. 8 is a view illustrating an example of a patient individual situation DB that is used in step SA5 in FIG. 4.

FIG. 9 is a view illustrating a configuration example of a medical information learning apparatus illustrated in FIG. 1.

FIG. 10 is a view illustrating a flow of a machine learning process by the medical information learning apparatus illustrated in FIG. 9.

FIG. 11 is a view schematically illustrating an update process relating to step SB3 in FIG. 10.

FIG. 12 is a view illustrating a configuration example of a medical information display apparatus illustrated in FIG. 1.

FIG. 13 is a view illustrating a flow of a medical knowledge graph display process by the medical information display apparatus illustrated in FIG. 12.

FIG. 14 is a view illustrating an example of an initial screen that is displayed in step SC1 in FIG. 13.

FIG. 15 is a view illustrating an example of a display screen that is displayed in step SC4 in FIG. 13.

FIG. 16 is a view illustrating a display example of a display graph relating to the entirety of patients.

FIG. 17 is a view illustrating a display example of a display graph relating to the entirety of diseases.

FIG. 18 is a conceptual view of allocation of graph features (order of occurrence, count of occurrences, and degree of occurrence) according to Applied Example 1.

FIG. 19 is a view schematically illustrating inputs and outputs of two machine learning models according to Applied Example 2.

FIG. 20 is a view schematically illustrating a convolution process by a graph convolution layer according to Applied Example 3.

FIG. 21 is a view illustrating an example of inputs and outputs of dense layers according to Applied Example 4.

FIG. 22 is a view schematically illustrating machine learning models according to Applied Example 5.

FIG. 23 is a view representing an outline of graph features according to Applied Example 6.

FIG. 24 is a view representing an outline of a graph feature according to Applied Example 7.

FIG. 25 is a view illustrating an example of an estimation process of medical judgment information according to Applied Example 7.

FIG. 26 is a view schematically illustrating a relationship between a patient graph and spatial information according to Applied Example 9.

FIG. 27 is a view illustrating a concept of a patient graph network.

FIG. 28 is a view representing an outline of graph features according to Applied Example 10.

### DETAILED DESCRIPTION

A medical information processing apparatus according to one embodiments include a processing circuitry. The processing circuitry obtains medical care information relating to medical care events of a target patient. The processing circuitry maps the medical care information on a first graph to generate a second graph relating to the target patient. The first graph includes nodes corresponding to the medical care events and edges indicative of a relationship between the nodes. The processing circuitry estimates medical judgment information relating to the target patient, based on the second graph relating to the target patient.

Hereinafter, referring to the accompanying drawings, embodiments of a medical information processing apparatus, a medical information learning apparatus, a medical information display apparatus, and a medical information processing method will be described in detail.

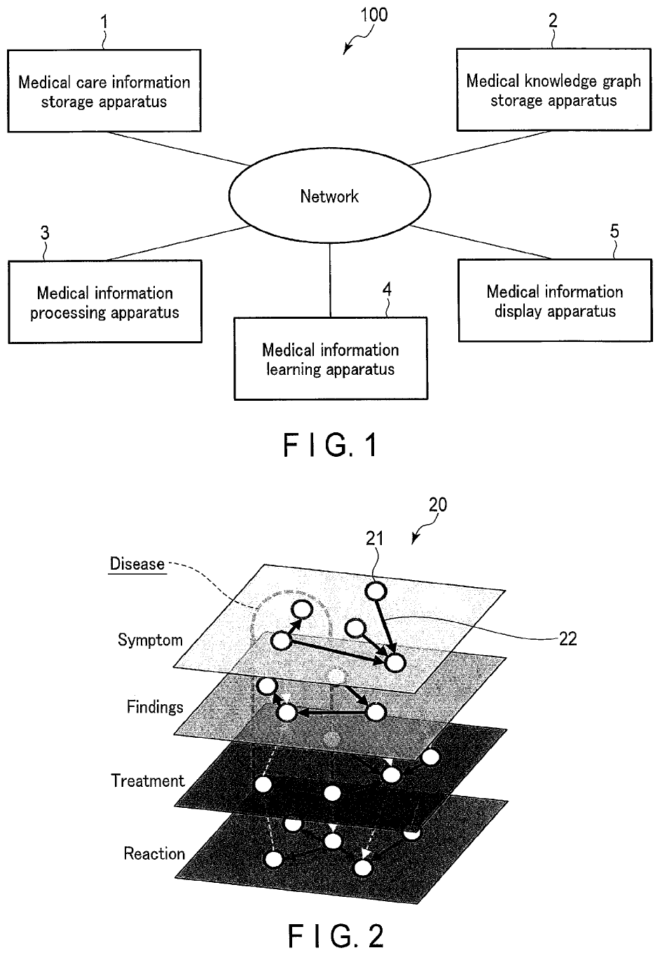

FIG. 1 is a view illustrating a configuration example of a medical information system 100 according to an embodiment. The medical information system 100 is a computer network system including a medical care information storage apparatus 1, a medical knowledge graph storage apparatus 2, a medical information processing apparatus 3, a medical information learning apparatus 4, and a medical information display apparatus 5. The medical care information storage apparatus 1, medical knowledge graph storage apparatus 2, medical information processing apparatus 3, medical information learning apparatus 4 and medical information display apparatus 5 are mutually communicably connected to a network.

The medical care information storage apparatus 1 is a computer including a storage device that stores medical care information or the like of a plurality of patients. The medical knowledge graph storage apparatus 2 is a computer including a storage device that stores a medical knowledge graph or the like. The medical knowledge graph is expressed by a graph structure that is generated based on medical care information of a plurality of patients, and medical knowledge such as medical ontology. Hereinafter, it is presupposed that the medical knowledge graph is a graph. The medical information processing apparatus 3 is a computer that estimates medical judgment information relating to a target patient by utilizing the medical care information of the target patient and the medical knowledge graph. The medical information processing apparatus 3 can also accumulate various kinds of medical knowledge in the medical knowledge graph. The medical information learning apparatus 4 is a computer that trains a machine learning model used in the estimation of the medical judgment information. The medical information display apparatus 5 is a computer that extracts desired medical knowledge from the medical knowledge graph, and displays the desired medical knowledge.

FIG. 2 is a conceptual view of a medical knowledge graph 20. The medical knowledge graph 20 is a graph in which medical knowledge and medical care information of patients are integrated. The medical care information is information generated by medical care for certain patients. The medical care information is classified into information relating to medical care events (hereinafter referred to as “medical care event information”) and information relating to diseases (hereinafter “disease information”). The medical care events are concrete items relating to medical care. The kinds of the medical care events are classified into, for example, four categories, i.e. a symptom, findings, a treatment and a reaction. The symptom is a change in mind and body occurring due to a disease. Specifically, the kinds of the medical care events relating to the symptom are, for example, swelling, palpitation, and difficulty in breathing. The findings are a doctor's judgment on the symptom. The findings may be further classified into a category of a physical finding and a category of an examination finding. The treatment is medical practice for curing or relieving the symptom. The kinds of the medical care events relating to the treatment are, for example, the administration of cardiotonic, CRT-embedding surgery, and the like. The reaction is a reaction of the mind and body of the patient to the treatment. The reaction may be further classified into a category of a treatment reaction and a category of a side effect. The kinds of the medical care events relating to the reaction are, for example, the presence of diuresis of 40 mL/h or more, or the presence of electrolyte abnormality, in connection with the administration of a diuretic drug. The disease information is information of the name or symbol relating to a disease that patient is diagnosed to contract.

The medical care information is collected by various hospital information systems, such as HIS (Hospital Information System), RIS (Radiology Information System), and PACS (Picture Archiving and Communication System). Each piece of the medical care event information and disease information included in the medical care information is correlated with the date of occurrence of the information.

As illustrated in FIG. 2, the medical knowledge graph 20 is a graph constituted by a plurality of nodes 21 and a plurality of edges 22. The nodes 21 correspond to medical care events. The edges 22 indicate the relationship between the nodes 21. Specifically, the edges 22 indicate the relationship between the medical care events corresponding to the connected nodes 21. The relationship between the medical care events means the cause-and-effect relation or the correlation between the medical care events. The cause-and-effect relation means the relation between the cause and effect, which exists between the medical care events, and the correlation means a relationship without a cause-and-effect relation. Specifically, the edge 22 indicative of the cause-and-effect relation is directed, and the edge 22 indicative of the correlation is undirected. The edge 22 can connect medical care events belonging to different categories. The medical care events used in the medical knowledge graph 20 are selected from the medical care information that is stored in the medical care information storage apparatus 1, based on the medical knowledge, medical ontology, and the like. The relationship between one medical care event and another medical care event is analyzed based on the medical knowledge, medical ontology, and the like, and, when the relationship is recognized, two nodes 21 corresponding to these two medical care events are connected by the edge 22.

Even in the case of the same disease, the presence/absence or the strength/weakness of the relationship between the medical care events varies depending on individual situations of individual patients. Even if the disease of the patient is estimated without considering the individual situation of the patient, a proper result cannot always be obtained.

The medical knowledge graph 20 does not include a node corresponding to a medical care event that is a disease. A certain specific disease is extracted from a set of a series of medical care events of the symptom, findings, treatment and reaction occurring in a target patient. In other words, the medical knowledge graph 20 expresses an individual disease by a chain of relationships of the series of medical care events of the symptom, findings, treatment and reaction. It can also be said that the medical knowledge graph 20 is a graph representing concepts of diseases, which describes the chain of relationships of the series of medical care events of the symptom, finding, treatment and reaction. The medical information system 100 precisely executes medical judgment such as disease estimation, by constructing a medical knowledge graph (patient graph) that adapts to the context that is the individual situation of the patient.

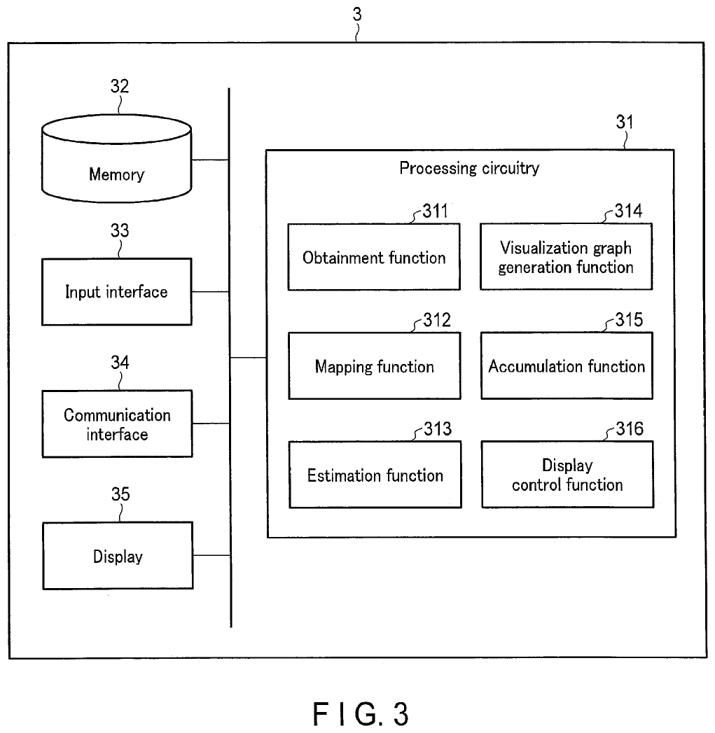

FIG. 3 is a view illustrating a configuration example of the medical information processing apparatus 3 according to the present embodiment. As illustrated in FIG. 3, the medical information processing apparatus 3 includes processing circuitry 31, a memory 32, an input interface 33, a communication interface 34 and a display 35.

The processing circuitry 31 includes processors such as a CPU (Central Processing Unit) and a GPU (Graphics Processing Unit). The processing circuitry 31 implements an obtainment function 311, a mapping function 312, an estimation function 313, a visualization graph generation function 314, an accumulation function 315 and a display control function 316, by executing a medical information processing program for estimating and presenting medical judgment information. Note that the functions 311 to 316 may not be implemented by single processing circuitry. A plurality of independent processors may be combined to constitute processing circuitry, and the processors may implement the functions 311 to 316 by executing programs. Besides, the functions 311 to 316 may be modularized programs that constitute a medical information processing program, or may be individual programs. The programs are stored in the memory 32.

By implementing the obtainment function 311, the processing circuitry 31 obtains various information. For example, the processing circuitry 31 obtains, from the medical care information storage apparatus 1, medical care event information that is medical care information relating to medical care events of a target patient. In addition, the processing circuitry 31 obtains a medical knowledge graph from the medical knowledge graph storage apparatus 2.

By implementing the mapping function 312, the processing circuitry 31 generates a patient graph relating to a target patient, in which the medical care event information of the target patient is mapped on a medical knowledge graph including nodes corresponding to the medical care events and edges representing the relationship between the nodes.

By implementing the estimation function 313, the processing circuitry 31 estimates medical judgment information relating to the target patient, based on the patient graph relating to the target patient. The medical judgment information is information relating to at least one medical judgment among disease classification information, prognosis estimation information, and severity level classification information.

By implementing the visualization graph generation function 314, the processing circuitry 31 generates a patient graph for display (hereinafter referred to as “visualization graph”), which relates to the target patient, based on the medical knowledge graph and the medical care information of the target patient.

By implementing the accumulation function 315, the processing circuitry 31 accumulates, in the medical knowledge graph, individual situation information relating to the target patient. The individual situation information will be described later.

By implementing the display control function 316, the processing circuitry 31 displays various information on the display 35. For example, the processing circuitry 31 displays the medical judgment information relating to the target patient, and the visualization graph of the target patient.

The memory 32 is a storage device storing various kinds of information, such as a ROM (Read Only Memory), a RAM (Random Access Memory), an HDD (Hard Disk Drive), an SSD (Solid State Drive), an integrated-circuit storage device, or the like. Aside from the above storage device, the memory 32 may be a drive unit that reads/writes various kinds of information from/to a portable storage medium such as a CD (Compact Disc), a DVD (Digital Versatile Disc) or a flash memory, or a semiconductor memory device. In addition, the memory 32 may be provided in another computer that is connected to the medical information processing apparatus 3 via a network. For example, the memory 32 stores the medical knowledge graph obtained by the obtainment function 311.

The input interface 33 accepts various input operations from an operator, converts the accepted input operations to electric signals, and outputs the electric signals to the processing circuitry 31. Specifically, the input interface 33 is connected to input devices such as a mouse, a keyboard, a trackball, a switch, a button, a joystick, a touch pad, and a touch-panel display. The input interface 33 outputs an electric signal, which corresponds to an input operation to the input device, to the processing circuitry 31. In addition, the input device connected to the input interface 33 may be an input device provided in another computer that is connected via a network or the like.

The communication interface 34 is an interface for transmitting/receiving various information to/from other computers such as the medical care information storage apparatus 1, medical knowledge graph storage apparatus 2, medical information learning apparatus 4 and medical information display apparatus 5 included in the medical information system 100.

The display 35 displays various information in accordance with the display control function 316 of the processing circuitry 31. As the display 35, for example, use can be made of, as appropriate, a liquid crystal display (LCD), a CRT (Cathode Ray Tube) display, an organic electro-luminescence (EL) display (OELD), a plasma display, or some other freely chosen display. Furthermore, a projector may be provided in place of, or in combination with, the display 35.

Next, a medical information process, which is executed by the processing circuitry 31 according to the medical information processing program, is described. In the embodiment below, it is assumed that the medical judgment information is the disease classification information.

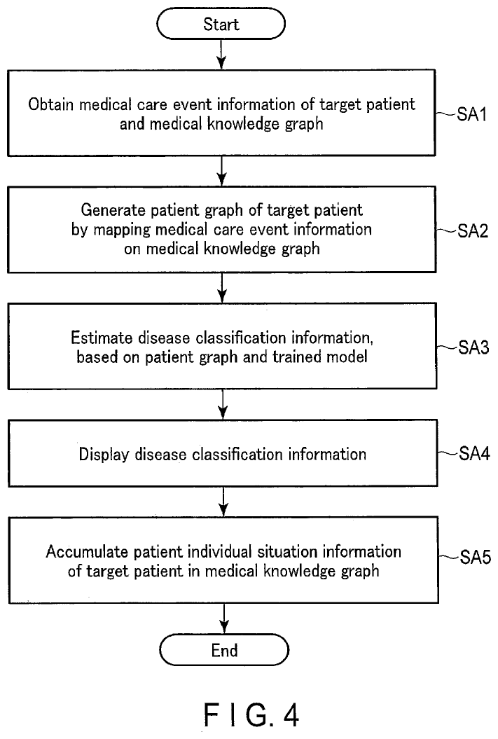

FIG. 4 is a view illustrating a flow of the medical information process. As illustrated in FIG. 4, by implementing the obtainment function 311, the processing circuitry 31 obtains the medical care event information of the target patient, and the medical knowledge graph (step SA1). The medical knowledge graph is obtained from the medical knowledge graph storage apparatus 2. The medical care event information is information including a combination between the kinds of medical care events and values indicative of the degrees of relevance of the target patient to the medical care events. The degree of relevance is typically defined by binary values, one indicating relevance, and the other indicating irrelevance. However, the degree of relevance may be defined by three or more discrete values or sequential values indicating the degree of relevance. In the description below, it is assumed that the degree of relevance in the medical care event information obtained in step SA1 is expressed by a binary value. For example, the medical care event information is obtained in a manner described below.

To start with, the processing circuitry 31 obtains a history of medical care information of a target patient from the medical care information storage apparatus 1. Then, the processing circuitry 31 obtains medical care event information from the history of the medical care information. For example, the processing circuitry 31 applies an information process, such as a document search with a search key being a medical care event registered in the medical knowledge graph, to the history of the medical care information of the target patient, and determines whether the medical care event registered in the medical knowledge graph is included in the history of the medical care information. Then, the processing circuitry 31 generates medical care event information in which a value indicative of the inclusion in the history, i.e. a value “1” indicative of relevance, or a value indicative of the non-inclusion in the history, i.e. a value “0” indicative of irrelevance, is allocated to each of the medical care events. Note that the medical care event information may be stored in the medical care information storage apparatus 1, and the medical care event information may be obtained from the medical care information storage apparatus 1 in step SA1. In step SA1, the medical care event information during a period corresponding to the period, which is taken into account when estimating the disease classification information, is obtained. In addition, in the medical care event information obtained in step SA1, medical care event information of all categories may not be included, and medical care event information of some categories may not be included.

If step SA1 is executed, the processing circuitry 31 generates, by implementing the mapping function 312, a patient graph of the target patient by mapping the medical care event information obtained in step SA1 on the medical knowledge graph (step SA2).

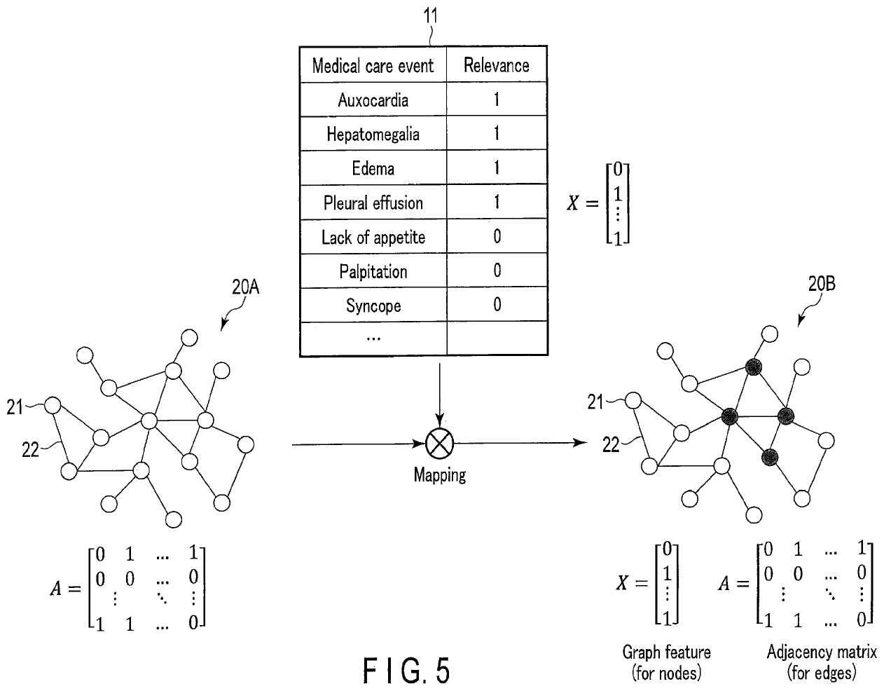

FIG. 5 is a view illustrating an example of the mapping process relating to step SA2. As illustrated in FIG. 5, the medical care event information includes information indicative of relevance or irrelevance to each of the medical care events. In medical care event information 11, the medical care events, for which the relevance or irrelevance is indicated, correspond to the nodes constituting the medical knowledge graph. When the medical care event occurs in regard to the target patient, a value “1” indicative of “relevance” is allocated to the medical care event. When the medical care event does not occur in regard to the target patient, a value “0” indicative of “irrelevance” is allocated to the medical care event. For example, the medical care event information 11 is given in such a form that the medical care event “auxocardia” is the relevance “1”, the medical care event “edema” is the relevance “1”, and the medical care event “palpitation” is the irrelevance “0”.

As described above, a medical knowledge graph 20A is constituted by nodes 21 and edges 22 connecting the nodes 21. The nodes 21 and edges 22 are also called “graph constituents”. The nodes 21 correspond to the medical care events that are defined in advance. The medical care events are classified into four categories of the symptom, findings, treatment and reaction, and concrete medical care events are allocated to the respective nodes 21. The edges 22 represent the relationship between medical care events. When two medical care events have a relation, two nodes 21 corresponding to the two medical care events are connected by the edge 22. When two medical care events have no relation, two nodes 21 corresponding to the two medical care events are not connected by the edge 22.

The edges 22 can be described by an adjacency matrix A indicated by equation (1) below. Rows and columns correspond to the nodes 21. For example, since a matrix element of a row number “1” and a column number “0” is “0”, it is indicated that there is an edge connecting the first node and the second node. Since a diagonal element of a row number and a column number that are equal represents an edge between identical nodes, “0” is allocated.

$$A = [ \begin{matrix}0 & 1 & … & 1 \\ 1 & 0 & … & 1 \\ ⋮ & & ⋱ & ⋮ \\ 1 & 1 & … & 0\end{matrix} ] \qquad ( 1 )$$

In the present embodiment, the edges are expressed by an adjacency matrix, but the form of expression is not limited to this. The edges may be expressed by other expression forms such as an adjacency list, or a kernel matrix (Gaussian kernel, or linear kernel) that expresses the similarity between nodes as a strength of the edge. In addition, although the medical knowledge graph according to the present embodiment is assumed to be an undirected graph in which the directions of arrows of edges are absent, i.e. in which there is no cause-and-effect relation between medical care events, the medical knowledge graph may be a directed graph in which the directions of arrows of edges are present, i.e. in which there is a cause-and-effect relation between medical care events.

As illustrated in FIG. 5, the processing circuitry 31 generates a patient graph 20B by mapping the medical care event information 11 on the medical knowledge graph 20A. Specifically, a feature (hereinafter “graph feature”) is allocated to each node. The graph feature has, for example, a binary value indicative of relevance or irrelevance of the medical care event corresponding to the node. For example, as illustrated in FIG. 5, since the medical care event “pleural effusion” is the relevance “1”, “1” is allocated to the graph feature of the node corresponding to the medical care event “pleural effusion”. Since the medical care event “syncope” is the irrelevance “0”, “0” is allocated to the graph feature of the node corresponding to the medical care event “syncope”. In FIG. 5, among the nodes 21 included in the patient graph 20B, the nodes to which the graph feature “1” is allocated are expressed by colored circles, and the nodes to which the graph feature “0” is allocated are expressed by white blank circles.

The graph feature X of each node can be expressed by a matrix, as indicated in equation (2) below. The elements in the column of the graph feature X correspond to the respective nodes. A value, such as relevance “1” or irrelevance “0”, is allocated to each element. As described above, the values of the graph feature X are not limited to binary values. It can also be said that the graph feature X is a matrix expression of the medical care event information 11.

$$X = [ \begin{matrix}0 \\ 1 \\ ⋮ \\ 1\end{matrix} ] \qquad ( 2 )$$

As will be described later, the graph feature is not limited to the relevance or irrelevance of the medical care events, and may include values indicative of the count of occurrences, the order of occurrence, the degree of occurrence, or the like, of medical care events.

Although it is presupposed that the medical knowledge graph in the above-described embodiment is common to a plurality of patients, the embodiment is not limited to this. For example, the medical knowledge graph storage apparatus 2 may store a plurality of medical knowledge graphs having different relations of connection of edges to nodes, i.e. different combinations of numerical values of the elements included in the adjacency matrix. In this case, the processing circuitry 31 selects, from the medical knowledge graphs, a medical knowledge graph that is used for the target patient. The selection of the medical knowledge graph may be manually performed by the user through the input interface 33, or may be automatically performed in accordance with a freely selected algorithm.

If step SA2 is executed, the processing circuitry 31 estimates, by implementing the estimation function 313, disease classification information, based on the patient graph generated in step SA2 and a trained model (step SA3). The trained model is a machine learning model in which learning parameters are trained by the medical information learning apparatus 4. The learning parameters are trainable parameters such as a weight, a bias, and the like.

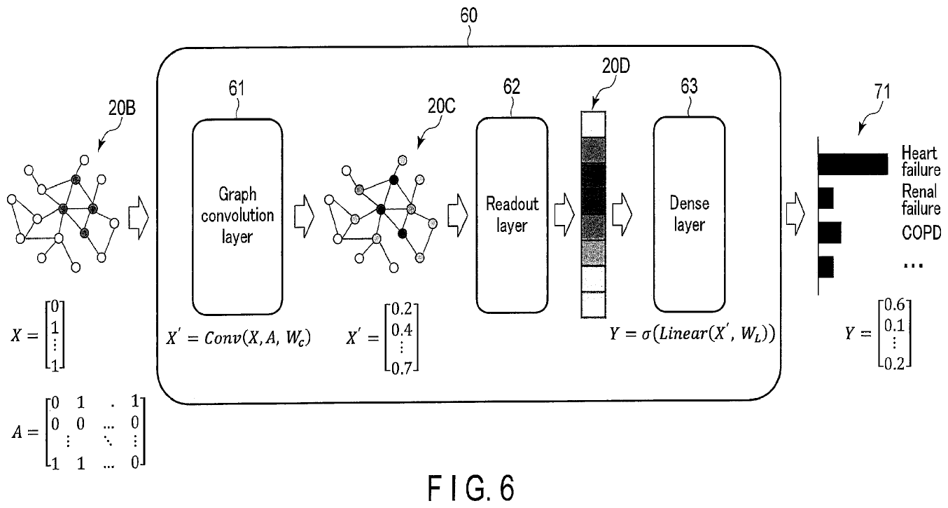

FIG. 6 is a view illustrating an example of an estimation process relating to step SA3. As illustrated in FIG. 6, the processing circuitry 31 estimates disease classification information 71 by applying the patient graph 20B to a trained model 60. The trained model 60 is a machine learning model which inputs therein the patient graph 20, and in which the learning parameters are trained so as to output the disease classification information 71.

As illustrated in FIG. 6, the trained model 60 includes a graph convolution layer 61, a readout layer 62 and a dense layer 63. The graph convolution layer 61 is a graph convolutional network (GCN) that inputs therein the patient graph 20B before convolution and outputs a patient graph 20C after convolution. The graph convolution layer 61 executes a graph convolution process on each node. Specifically, the graph convolution layer 61 executes a convolution operation, based on the graph feature before convolution of a process-target node, the graph feature of a node (hereinafter referred to “adjacent node) that is connected to the process-target node via an edge, and a weight on the edge connecting the process-target node and the adjacent node, and computes a graph feature after convolution of the process-target node. The weight on the edge is a weight parameter included in a weight matrix of the graph convolution layer 61. Hereinafter, the weight on the edge is called “GCN weight”.

A graph feature X′ after convolution of the process-target node can be expressed, as indicated in equation (3) below, as a function Conv based on the graph feature X before convolution of the process-target node, the adjacency matrix A, and a GCN weight W_{C}. The GCN weight W_{C} is a learning parameter that is trained by the medical information learning apparatus 4. The number of layers of the graph convolution layer 61 may be one or more. As will be described later, in the graph convolution layer 61, filters may be separated for individual medical care event categories, individual directions of cause-and-effect, and individual strengths of cause-and-effect and correlation. The convolution process indicated in equation (3) or the like is repeated by a number corresponding to the number of layers. The patient graph 20C after convolution is generated by executing the above-described convolution operation with respect to each node included in the patient graph 20B before convolution. Note that I is a unit matrix, and A is a degree matrix of the graph.

$${X}^{′} = Conv ( X , A , {W}_{c} ) = {Λ}^{- \frac{1}{2}} \hat{A} {Λ}^{- \frac{1}{2}} X {W}_{c} \hat{A} = A + I \qquad ( 3 )$$

As illustrated in FIG. 6, as a result of the convolution operation, the graph feature X′ after convolution does not have a binary value such as value “1” or value “0”, but has a continuous value indicative of the degree of relevance, such as “0.2” or “0.4”.

The readout layer 62 is a network layer that converts the patient graph 20C after convolution to a feature vector 20D. The feature vector 20D is one column vector having the same number of dimensions as the number of nodes included in the patient graph 20C after convolution. Specifically, the readout layer 62 reads the graph feature of each node included in the patient graph 20C after convolution, and converts the graph feature to the feature vector 20D.

The dense layer 63 is a network layer that converts the feature vector 20D to the disease classification information 71. The dense layer 63 is also called “multilayer perceptron (MLP)”. The dense layer 63 executes a class classification task or a regression task. The class classification task may be a 2-class classification for determining the presence/absence of the relevant disease, or a multi-class classification for specifying one disease from a plurality of disease candidates. The class classification task may be a multi-label classification which allows a plurality of labels for an identical data set. The regression task outputs a numerical value indicative of the probability of relevance to each of one or more relevant diseases. The dense layer 63 is also referred to as fully-connected layer, linear layer, or multilayer perceptron (MLP).

In the present embodiment, the dense layer 63 includes a classifier that executes multi-class classification for classifying the feature vector 20D into a plurality of classes corresponding to a plurality of diseases, respectively. In the multi-class classification, an operation of a softmax function, which outputs the probability that the feature vector 20D belongs to each disease (class), is executed. The probability that the feature vector 20D belongs to each disease is output as the disease classification information 71. For example, as illustrated in FIG. 6, the probability of relevance to the disease “heart failure”, the probability of relevance to the disease “renal failure”, the probability of relevance to the disease “COPD”, and the like are output as the disease classification information 71. In this case, the disease classification information 71 can be expressed by a matrix notation Y, as indicated in equation (4) below.

$$Y = [ \begin{matrix}0.6 \\ 0.1 \\ ⋮ \\ 0.2\end{matrix} ] \qquad ( 4 )$$

A relevance probability Y of each disease in the disease classification information 71 is computed based on the graph feature X′ after convolution, and a disease weight W_{L}, as indicated in equation (5) below. The disease weight W_{L} is a learning parameter that is trained by the medical information learning apparatus 4. The disease weight W_{L} is set for each class in regard to each element (node) of the feature vector 20D. Specifically, it can also be said that the disease weight W_{L} is a parameter indicative of an influence level to the disease of each node. The relevance probability Y of each disease can be obtained by applying an activation function σ to a weighted addition operation Linear by the disease weight W_{L} of the graph feature X′. The activation function σ is implemented by a softmax function or the like, as described above.

Y=σ(Linear(X′,W_{L})) (5)

If step SA3 is executed, the processing circuitry 31 displays, by implementing the display control function 316, the disease classification information estimated in step SA3 (step SA4). In step SA4, the processing circuitry 31 displays the disease classification information by a display screen of a predetermined layout.

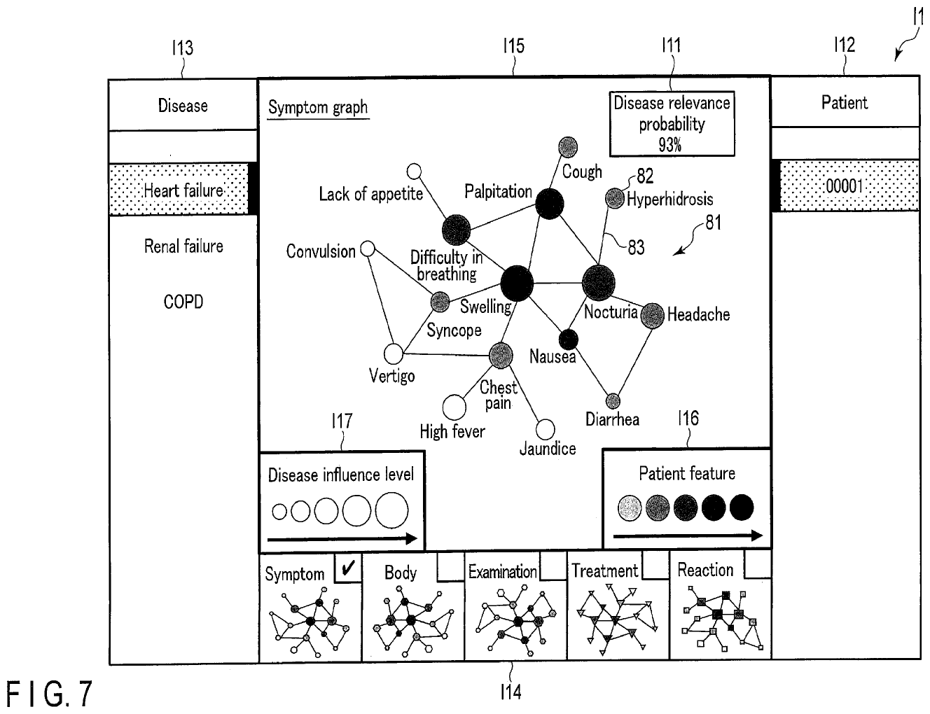

FIG. 7 is a view illustrating an example of a display screen I1 of disease classification information. As illustrated in FIG. 7, the display screen I1 includes a display field I11 of disease classification information, a selection field I12 of a patient, a selection field I13 of a disease, a selection field I14 of a medical care event category, and a display field I15 of a patient graph. The display field I11 of disease classification information displays the disease classification information estimated in step SA3. The selection field I12 of a patient displays an identifier of a target patient (hereinafter referred to as “patient identifier”). As the patient identifier, a patient ID, a patient name or the like is used. The patient identifier displayed in the selection field I12 can be selected by the operator through the input interface 33 or the like. The information corresponding to the selected patient identifier is displayed on the display screen I1.

The selection field I13 of a disease displays an identifier of a target disease (hereinafter referred to as “disease identifier”). As the disease identifier, the name, symbol or the like of a disease is used. The disease identifier displayed in the selection field I13 can be selected by the operator through the input interface 33 or the like. The relevance probability corresponding to the selected disease identifier is displayed in the display field I11.

The selection field I14 of a medical care event category displays a list of medical care event categories of display targets. A medical care event category that is displayed may be the name or symbol of the medical care event category, or a simulated image or a thumbnail image of the patient graph relating to the medical care event category.

The display field I15 of a patient graph displays a visualization graph 81 based on a patient graph before a convolution process or a patient graph after the convolution process. Hereinafter, a detailed description is given of the generation of the visualization graph 81 by the visualization graph generation function 314 and the display of the visualization graph 81 by the display control function 316. Note that when the patient graph before the convolution process and the patient graph after the convolution process are not particularly distinguished, these patient graphs are simply referred to as “patient graph”.

To start with, the processing circuitry 31 specifies the medical care event category selected in the selection field I14. When the medical care event category is not selected in the selection field I14, or the visualization graph 81 is displayed by default, all medical care event categories are specified as selected medical care event categories. Then, the processing circuitry 31 extracts, from the patient graph, a graph (hereinafter referred to as “partial patient graph”) which is composed of nodes belonging to the specified medical care event category and edges connecting the nodes. For example, in FIG. 7, since the symptom category is selected in the selection field I14, a partial patient graph, which is composed of nodes belonging to the symptom category and edges connecting the nodes, is extracted from the patient graph.

Next, the processing circuitry 31 simultaneously or successively determines a patient feature and a disease influence level with respect to each of the nodes belonging to the specified medical care event category. The patient feature is information in which the graph feature of the target patient in regard to each node is expressed as a scalar value. When the graph feature is a scalar value, the patient feature is determined to be the scalar value or a value obtained by correcting the scalar value. When the graph feature is a vector, the graph feature vector is first converted to a scalar value such as a statistical value of the element value of the vector. The statistical value may be set to be, for example, an average value, a median, a maximum value, a minimum value, an arbitrary quantile, or the like of the element values of the graph feature vector. The patient feature is determined to be a scalar value based on the graph feature vector, or a value obtained by correcting the scalar value. In order to obtain the scalar value, use may be made of a machine learning model that computes the scalar value from the graph feature vector. Note that the patient feature may be determined based on the graph feature X before convolution.

The disease influence level is information in which the influence level of the target disease on each node is expressed as a numerical value. The disease influence level is determined based on the disease weight W_{L} of each node. As described above, the disease weight W_{L} is set for each class in regard to each element (node) of the feature vector. When the dense layer 63 is a single layer, the disease influence level is determined to be a value agreeing with the disease weight W_{L} or a value based on the disease weight W_{L}. When the dense layer 63 is a multilayer, the contribution ratio of each node is computed based on the disease weight W_{L} of each layer. For example, the contribution ratio may be calculated by learning a contribution ratio for explanation by using a comprehensive or local explainable model based on the dense layer 63. The disease influence level is determined to be a value agreeing with the contribution ratio, or a value based on the contribution ratio.

Next, based on the patient feature and the disease influence level, the processing circuitry 31 determines a display mode of nodes. An example of the display mode is described below. As illustrated in FIG. 7, the display color of the node is determined in accordance with the patient feature. As the patient feature increases, the display color is determined in such a manner as to gradually change from light red to deep red. Auxiliary information 116, which indicates to the operator the visual relationship between the patient feature and the display color, is displayed. The display size of the node is determined in accordance with the disease influence level. The display size is determined in such a manner as to gradually change from a small size to a large size as the disease influence level increases. Auxiliary information 117, which indicates to the operator the visual relationship between the influence level to the disease and the display size, is displayed.

In addition, the processing circuitry 31 displays the disease relevance probability of the target disease in the display field I11, and displays, in the display field I15, the visualization graph 81 that visualizes the partial patient graph. The visualization graph 81 represents the partial patient graph relating to the target patient designated in the selection field I12, the target disease selected in the selection field I13, and the medical care event belonging to the medical care event category selected in the selection field I14. For example, in FIG. 7, the visualization graph 81, which relates to the target patient “0001”, the target disease “heart failure” and the medical care event category “symptom”, is displayed. The processing circuitry 31 displays the nodes 82 included in the visualization graph 81, in a display mode corresponding to the patient features allocated to the nodes included in the patient graph. In addition, the processing circuitry 31 displays the nodes 82 included in the visualization graph 81, in a display mode corresponding to the disease influence levels allocated to the nodes included in the patient graph. The patient features and the disease influence levels are visualized in the nodes 82. Between nodes 82 having a relationship, an edge 83 connecting the nodes 82 is depicted. It is preferable that each node 82 is accompanied with the name or symbol of the medical care event corresponding to the node 82. Thereby, the meaning of the node can be understood.

The visualization graph is a display object that visualizes the patient graph which represents a series of medical care events occurring in the target patient by the nodes connected by edges in accordance with a mutual relationship. Each node is emphasized by the patient feature and disease influence level, which are unique to the target patient. By observing the visualization graph, the user can visually recognize what medical care event is involved in the development of the target disease. For example, the user can understand the medical care events of the “symptom” relating to the target disease “heart failure”, and the relationship between the medical care events, and can understand the degree of the patient feature of each medical care event and the degree of the disease influence level. Moreover, by observing the visualization graph, the user can understand the essential feature of the disease. Besides, the understanding of the mechanism of the disease is promoted.

At a freely chosen time point, the user can instruct a change of the medical care event category of the display target in the selection field I14. When a change of the medical care event category is instructed, the processing circuitry 31 reconstructs and displays, according to the same method as described above, the visualization graph based on the nodes of the medical care events belonging to the medical care event category after the change and the partial patient graph connecting the nodes. If a change of the target disease in the selection field I13 is instructed, the processing circuitry 31 re-computes, according to the same method as described above, the disease influence level, based on the disease weight W_{L} of the target disease after the change, and displays the nodes 82 in the display mode corresponding to the disease influence level.

If step SA4 is executed, the processing circuitry 31 accumulates, by implementing the accumulation function 315, the individual situation information of the target patient in the medical knowledge graph (step SA5). The individual situation information includes, for example, the graph feature before convolution of each node, the graph feature after convolution, the disease weight and the disease influence level. For example, the individual situation information is recorded in a database such as an LUT (Look Up Table). Hereinafter, this database is referred to as “patient individual situation DB”.

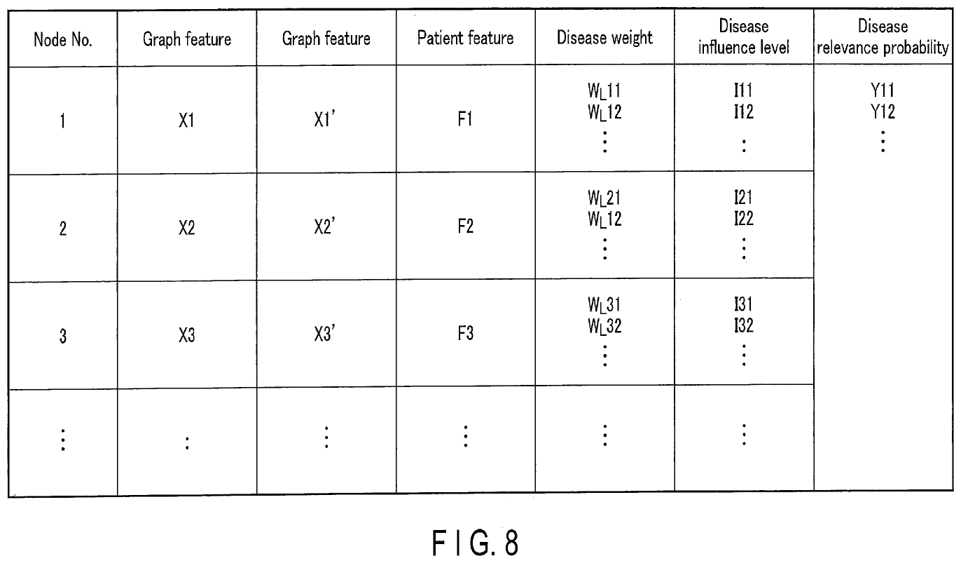

FIG. 8 is a view illustrating an example of the patient individual situation DB. As illustrated in FIG. 8, in the patient individual situation DB, in regard to each of the nodes included in the medical knowledge graph, such pieces of individual situation information, as the graph feature before convolution, the graph feature after convolution, the patient feature, the disease weight and the disease influence level, are correlated such that these pieces of individual situation information can be searched. In addition, in the patient individual situation DB, the disease relevance probability is correlated with the entirety of the patient individual situation information of the target patient. The individual situation information is computed by the processing circuitry 31, as described above. Note that the disease weight and the disease influence level are computed for each of a plurality of disease classes in regard to one node. For example, in regard to the node number “1”, the following is recorded: the graph feature “X1”, the graph feature “X1′” after convolution, the patient feature “F1”, a disease weight “W_{L}11” of a first disease label, a disease weight “W_{L}12” of a second disease label, . . . , a disease influence level “I11” of the first disease label, a disease influence level “I12” of the second disease label, . . . . Moreover, a disease relevance probability “Y11” of the first disease label, a disease relevance probability “Y12” of the second disease label, and the like are recorded for the entirety of the individual situation information. The disease label means a numerical value representative of the relevance of each disease class.

The patient individual situation DB is created for each of patients. The patient individual situation DB, together with the medical knowledge graph, is stored by the medical knowledge graph storage apparatus 2. Note that the individual situation information recorded in the patient individual situation DB may be part of the above-described information, or may include other information.

If step SA5 is executed, the medical information process by the processing circuitry 31 ends.

As described above, the medical information processing apparatus 3 according to the present embodiment includes the processing circuitry 31. The processing circuitry 31 obtains the medical care event information of the target patient. The processing circuitry 31 generates the patient graph relating to the target patient by mapping the medical care event information on the medical knowledge graph that includes the nodes corresponding to the medical care event information, and the edges representing the relationship between the nodes. The processing circuitry 31 estimates the medical judgment information relating to the target patient, based on the patient graph relating to the target patient.

Since the patient graph is the medical knowledge graph in which the medical care event information of the target patient is mapped, the patient graph reflects the individual situation of the patient. Since this patient graph is used, the medical judgment information, such as the disease relevance probability, can be estimated by taking into account the individual situations of individual patients. Furthermore, the support for medical judgment is enabled with robustness, without being affected by a specific symptom or a similar patient.

Next, the medical information learning apparatus 4 according to the present embodiment is described.

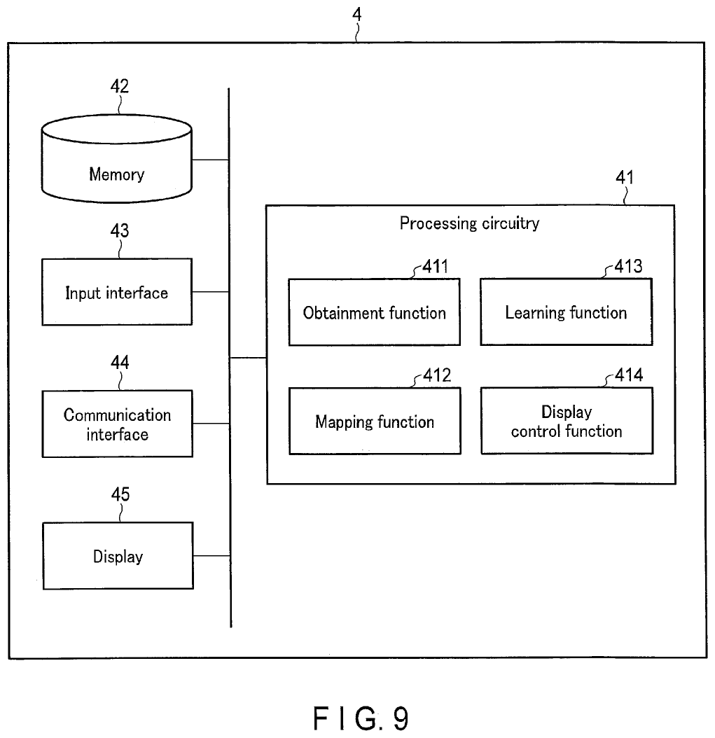

FIG. 9 is a view illustrating a configuration example of the medical information learning apparatus 4. As illustrated in FIG. 9, the medical information learning apparatus 4 includes processing circuitry 41, a memory 42, an input interface 43, a communication interface 44 and a display 45.

The processing circuitry 41 includes processors such as a CPU and a GPU. The processing circuitry 41 implements an obtainment function 411, a mapping function 412, a learning function 413 and a display control function 414, by executing a learning program for generating a trained model that the medical information processing apparatus 3 uses. Note that the functions 411 to 414 may not be implemented by single processing circuitry. A plurality of independent processors may be combined to constitute processing circuitry, and the processors may implement the functions 411 to 414 by executing programs. Besides, the functions 411 to 414 may be modularized programs that constitute a learning program, or may be individual programs. The programs are stored in the memory 42.

By implementing the obtainment function 411, the processing circuitry 41 obtains various information. For example, the processing circuitry 41 obtains, from the medical care information storage apparatus 1, a plurality of training samples relating to a plurality of patients. The training samples correspond to the medical care information, and include medical care event information and medical judgment information. The medical care event information is used as an input sample of machine learning, and the medical judgment information is used as an output sample (teaching sample) of machine learning. Note that the medical judgment information included in the training sample is not the medical judgment information estimated by the medical information processing apparatus 3, but information relating to a medical judgment decided by a doctor or the like for the patient. In addition, the processing circuitry 41 obtains a medical knowledge graph from the medical knowledge graph storage apparatus 2.

By implementing the mapping function 412, the processing circuitry 41 generates a patient graph relating to a training sample, by mapping the medical care information of the training sample on the medical knowledge graph including nodes corresponding to the medical care events and edges representing the relationship between the nodes.

By implementing the learning function 413, the processing circuitry 41 trains a machine learning model, based on the patient graph and the medical judgment information, and generates a trained model that estimates medical judgment information from the patient graph.

By implementing the display control function 414, the processing circuitry 41 displays various information on the display 45. For example, the processing circuitry 41 displays a setting screen of machine learning, or the like.

The memory 42 is a storage device storing various kinds of information, such as a ROM, a RAM, an HDD, an SSD, an integrated-circuit storage device, or the like. Aside from the above storage device, the memory 42 may be a drive unit that reads/writes various kinds of information from/to a portable storage medium such as a CD, a DVD or a flash memory, or a semiconductor memory device. In addition, the memory 42 may be provided in another computer that is connected to the medical information learning apparatus 4 via a network. For example, the memory 42 stores the medical knowledge graph obtained by the obtainment function 411.

The input interface 43 accepts various input operations from an operator, converts the accepted input operations to electric signals, and outputs the electric signals to the processing circuitry 41. Specifically, the input interface 43 is connected to input devices such as a mouse, a keyboard, a trackball, a switch, a button, a joystick, a touch pad, and a touch-panel display. The input interface 43 outputs an electric signal, which corresponds to an input operation to the input device, to the processing circuitry 41. In addition, the input device connected to the input interface 43 may be an input device provided in another computer that is connected via a network or the like.

The communication interface 44 is an interface for transmitting/receiving various information to/from other computers such as the medical care information storage apparatus 1, medical knowledge graph storage apparatus 2, medical information processing apparatus 3 and medical information display apparatus 5 included in the medical information system 100.

The display 45 displays various information in accordance with the display control function 414 of the processing circuitry 41. As the display 45, for example, use can be made of, as appropriate, a liquid crystal display, a CRT display, an organic EL display, a plasma display, or some other freely chosen display. Furthermore, a projector may be provided in place of, or in combination with, the display 45.

Next, a machine learning process, which is executed by the processing circuitry 41 according to the learning program, is described. In the embodiment below, it is assumed that the medical judgment information is the disease classification information.

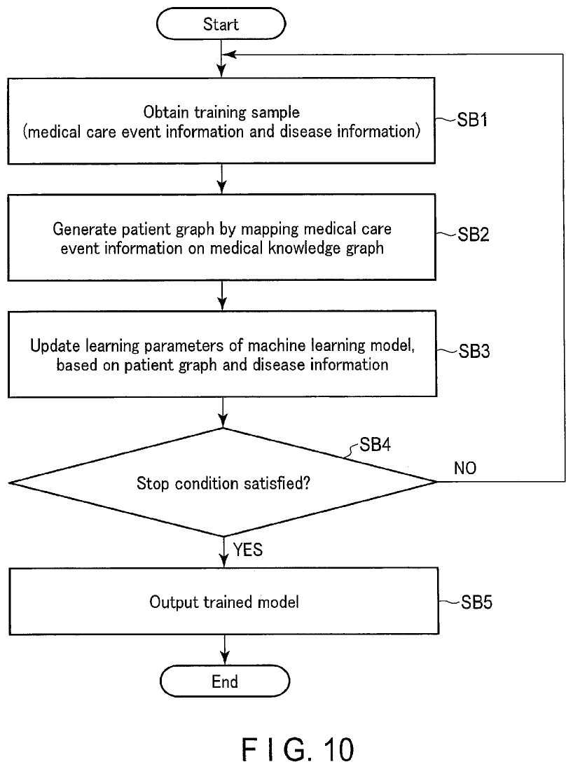

FIG. 10 is a view illustrating a flow of the machine learning process. As illustrated in FIG. 10, by implementing the obtainment function 411, the processing circuitry 41 obtains a training sample (step SB1). The training sample includes a combination of medical care event information and disease information in regard to one patient. The medical care event information, as described above, is the medical care information relating to the medical care events occurring in the patient. The disease information is information relating to disease that the one patient contracts, and corresponds to the medical care event information relating to the one patient. The disease information has a number of dimensions, which corresponds to the number of disease classes, and has values indicative of relevance or irrelevance in regard to the respective disease classes. The disease information is disease classification information to which values are added by humans, and is a kind of disease classification information. A value indicative of relevance of each disease class is also called “disease label”. In regard to the medical care event information relating to one patient, a disease label relating to one disease may be given, or a plurality of disease labels relating to two or more diseases may be given. In regard to the medical care event information relating to the one patient, two or more disease labels may be given in accordance with a hierarchical structure in nosology. The processing circuitry 41 may specify disease information, based on at least one of medical care event information and medical ontology.

If step SB1 is executed, the processing circuitry 41 generates a patient graph by mapping the medical care event information obtained in step SB1 on the medical knowledge graph (step SB2). Step SB2 is similar to step SA2.

If step SB2 is executed, the processing circuitry 41 updates learning parameters of the machine learning model, based on the patient graph generated in step SB2 and the disease information obtained in step SB1 (step SB3).

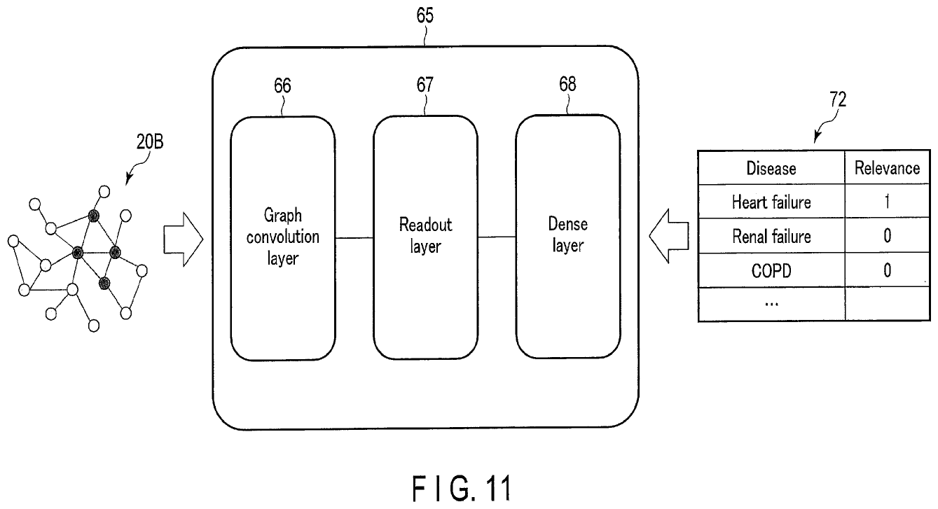

FIG. 11 is a view schematically illustrating an update process. As illustrated in FIG. 11, a machine learning model 65 is trained based on supervised learning in which a patient graph 20B is an input sample and disease information 72 is a teaching sample. The machine learning model 65 includes a graph convolution layer 66, a readout layer 67 and a dense layer 68. The graph convolution layer 66 corresponds to the graph convolution layer 61 of the trained model 60, and is a network layer that executes convolution process on the patient graph 20B. The readout layer 67 corresponds to the readout layer 62 of the trained model 60, and is a network layer that converts the patient graph to a feature vector. The dense layer 68 corresponds to the dense layer 63 of the trained model 60, and is a network layer that converts the feature vector to disease classification information. Initially, the learning parameters included in the machine learning model 65 are set to arbitrary default values. The learning parameters include GCN weights W_{C} of the graph convolution layer 66 and disease weights W_{L} of the dense layer 68.

In the update process, the processing circuitry 41 computes a loss function. The loss function is a function that evaluates an error between the disease classification information, which is computed by successively propagating the patient graph through the graph convolution layer 66, readout layer 67 and dense layer 68, and the disease information that is the teaching sample. The processing circuitry 41 updates the learning parameters of the machine learning model 65 in such a manner as to minimize the loss function in accordance with a freely selected optimization method. Concretely, as the learning parameters, the GCN weights We of the graph convolution layer 66 and the disease weights W_{L} of the dense layer 68 are updated. As the optimization method, use may be made of a freely selected method, such as stochastic gradient decent or Adam (adaptive moment estimation).

If step SB3 is executed, the processing circuitry 41 determines whether a stop condition is satisfied (step SB4). For example, the stop condition may be set such that the number of times of update of learning parameters reaches a predetermined number, or that the quantity of update of learning parameters is less than a threshold. If it is determined that the stop condition is not satisfied (step SB4: NO), the processing circuitry 41 obtains another training sample (step SB1). Then, similarly, with respect to the another training sample, the processing circuitry 41 successively executes the generation of the patient graph (step SB2), the update of the learning parameters (step SB3) and the determination as to whether the stop condition is satisfied (step SB4). In this manner, the processing circuitry 41 repeats the generation of the patient graph in regard to each training sample (step SB2), the update of the learning parameters (step SB3) and the determination as to whether the stop condition is satisfied (step SB4), until the stop condition is satisfied.

Then, if it is determined in step SB4 that the stop condition is satisfied (step SB4: YES), the processing circuitry 41 outputs, as a trained model, the machine learning model at a time point when the stop condition is satisfied (step SB5). The trained model is transmitted to and stored in the medical information processing apparatus 3 or the like.

If step SB5 is executed, the machine learning process by the processing circuitry 41 ends.

In the learning process in FIG. 10, it is assumed that the learning parameters of the machine learning model are updated in units of one training sample. However, the present embodiment is not limited to this. For example, minibatch training may be executed in which the learning parameters of the machine learning model are updated in units of a plurality of training samples. Besides, when the stop condition is that the quantity of update of learning parameters reaches less than the threshold, step SB4 may be executed following step SB2, and step SB3 may be executed when the stop condition is not satisfied.

As described above, the medical information learning apparatus 4 according to the present embodiment includes the processing circuitry 41. The processing circuitry 41 obtains the medical knowledge graph including nodes corresponding to medical care events and edges representing the relationship between the nodes, and the medical care information; generates the patient graph in which the medical care event information is mapped on the medical knowledge graph; and trains the machine learning model for estimating medical judgment information from the patient graph, based on the patient graph and the medical judgment information corresponding to the medical care event information.

According to the above-described configuration, since the machine training model for estimating the medical judgment information from the patient graph can be generated, the medical judgment information with high accuracy can be estimated.

Next, the medical information display apparatus 5 according to the present embodiment is described.

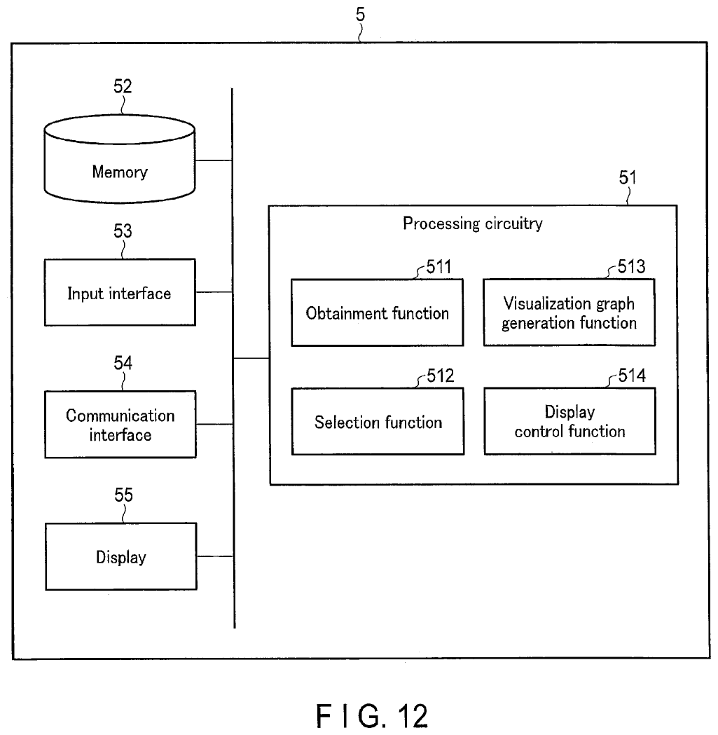

FIG. 12 is a view illustrating a configuration example of the medical information display apparatus 5. As illustrated in FIG. 12, the medical information display apparatus 5 includes processing circuitry 51, a memory 52, an input interface 53, a communication interface 54 and a display 55.

The processing circuitry 51 includes processors such as a CPU and a GPU. The processing circuitry 51 implements an obtainment function 511, a selection function 512, a visualization graph generation function 513 and a display control function 514, by executing a display program for displaying a medical knowledge graph. Note that the functions 511 to 514 may not be implemented by single processing circuitry. A plurality of independent processors may be combined to constitute processing circuitry, and the processors may implement the functions 511 to 514 by executing programs. Besides, the functions 511 to 514 may be modularized programs that constitute a display program, or may be individual programs. The programs are stored in the memory 52.

By implementing the obtainment function 511, the processing circuitry 51 obtains various information. For example, the processing circuitry 51 obtains the medical knowledge graph and the patient individual situation DB from the medical knowledge graph storage apparatus 2. As described above, the patient individual situation DB is a database in which, in regard to each of the nodes, such pieces of individual situation information, as the graph feature, the patient feature, the disease weight and the disease influence level, are correlated

By implementing the selection function 512, the processing circuitry 51 selects the patient and/or disease of the display target. The processing circuitry 51 may select only the patient, or may select only the disease, or may select both of the patient and the disease. Besides, one patient may be selected, or patients may be selected. Similarly, one disease may be selected, or diseases may be selected.

By implementing the visualization graph generation function 513, the processing circuitry 51 generates, based on the medical knowledge graph, a visualization graph that visualizes the patient graph in accordance with the patient feature and/or disease influence level relating to the patient and/or disease of the display target.

By implementing the display control function 514, the processing circuitry 51 displays various information on the display 55. For example, the processing circuitry 51 displays a visualization graph that visualizes the patient graph in accordance with the patient feature and/or disease influence level relating to the patient and/or disease of the display target. When a specific patient is selected as the display target, the processing circuitry 51 displays nodes included in the visualization graph, in a display mode corresponding to the patient feature of the specific patient, which corresponds to the nodes. When a specific disease is selected as the display target, the processing circuitry 51 displays nodes included in the visualization graph, in a display mode corresponding to the disease influence level of the specific disease, which corresponds to the nodes. When a combination of a specific patient and a specific disease is selected as the display target, the processing circuitry 51 displays nodes included in the visualization graph, in a first display mode corresponding to the patient feature of the combination, which corresponds to the nodes, and in a second display mode corresponding to the disease influence level of the combination, which corresponds to the nodes.

The memory 52 is a storage device storing various kinds of information, such as a ROM, a RAM, an HDD, an SSD, an integrated-circuit storage device, or the like. Aside from the above storage device, the memory 52 may be a drive unit that reads/writes various kinds of information from/to a portable storage medium such as a CD, a DVD or a flash memory, or a semiconductor memory device. In addition, the memory 52 may be provided in another computer that is connected to the medical information display apparatus 5 via a network. For example, the memory 52 stores the medical knowledge graph and patient individual situation DB obtained by the obtainment function 511.

The input interface 53 accepts various input operations from an operator, converts the accepted input operations to electric signals, and outputs the electric signals to the processing circuitry 51. Specifically, the input interface 53 is connected to input devices such as a mouse, a keyboard, a trackball, a switch, a button, a joystick, a touch pad, and a touch-panel display. The input interface 53 outputs an electric signal, which corresponds to an input operation to the input device, to the processing circuitry 51. In addition, the input device connected to the input interface 53 may be an input device provided in another computer that is connected via a network or the like.

The communication interface 54 is an interface for transmitting/receiving various information to/from other computers such as the medical care information storage apparatus 1, medical knowledge graph storage apparatus 2, medical information processing apparatus 3 and medical information learning apparatus 4 included in the medical information system 100.

The display 55 displays various information in accordance with the display control function 514 of the processing circuitry 51. As the display 55, for example, use can be made of, as appropriate, a liquid crystal display, a CRT display, an organic EL display, a plasma display, or some other freely chosen display. Furthermore, a projector may be provided in place of, or in combination with, the display 55.

Next, a medical knowledge graph display process, which is executed by the processing circuitry 51 according to the display program, is described. The medical knowledge graph display process is a process for viewing the individual situation information accumulated in the medical knowledge graph by the medical information processing apparatus 3, together with the medical knowledge graph.

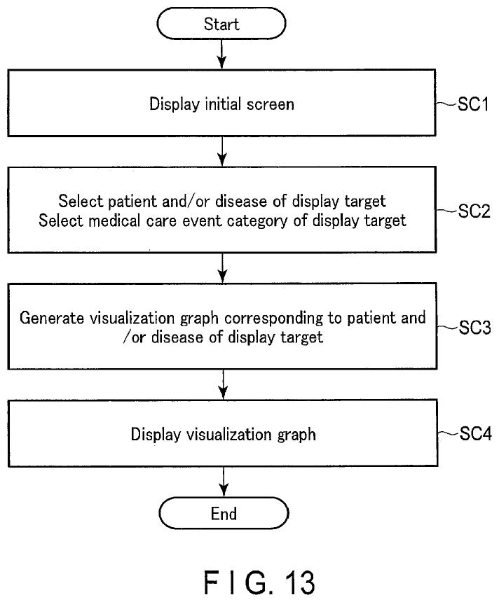

FIG. 13 is a graph illustrating a flow of the medical knowledge graph display process. It is assumed that at a start time point in FIG. 13, the processing circuitry 51 already obtains, from the medical knowledge graph storage apparatus 2, the medical knowledge graph and the patient individual situation DB relating to a plurality of patients. The medical knowledge graph and the patient individual situation DB relating to patients are stored in the memory 52.

As illustrated in FIG. 13, by implementing the display control function 514, the processing circuitry 51 displays an initial screen (step SC1). The initial screen is displayed on the display 55.

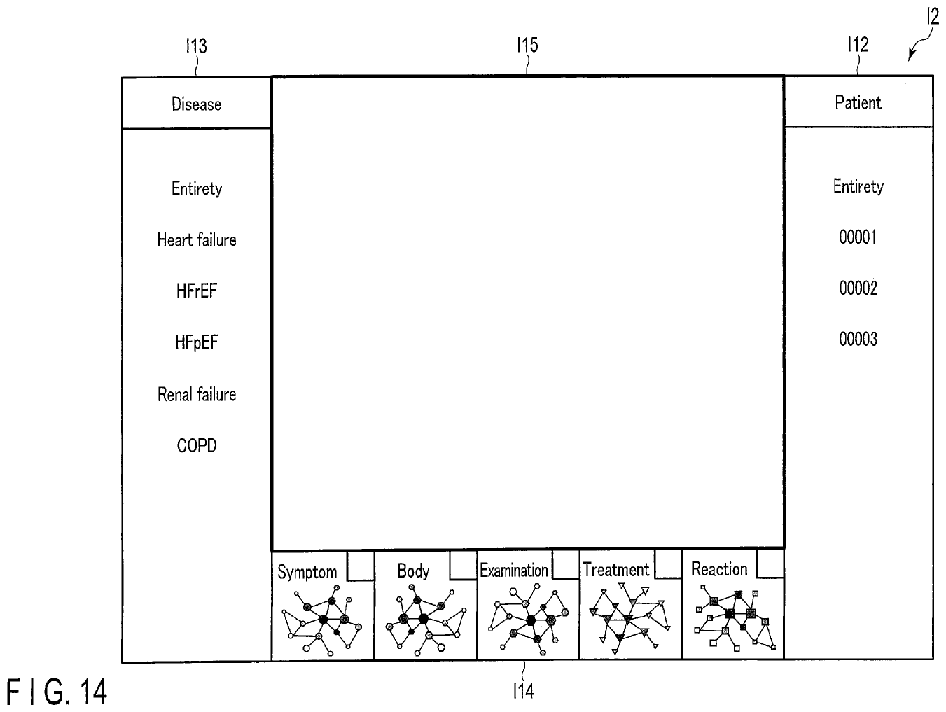

As illustrated in FIG. 14, an initial screen 12 includes a selection field I12 of a patient, a selection field I13 of a disease, a selection field I14 of a medical care event category, and a display field I15 of a patient graph. The selection field I12 selectably displays patient identifiers of a plurality of patients, whose individual situation information is accumulated in the medical knowledge graph. In addition, the selection field I12 may also selectably display a patient identifier indicative of a statistical group relating to patients. As the statistical group relating to patients, use can be made of, as appropriate, various classifications such as the entirety of patients whose individual situation information is accumulated in the medical knowledge graph, a classification based on gender, such as male or female, of the patients, and a classification based on age, such as sixties or seventies. In FIG. 14, by way of example, “entirety” indicative of the entirety of patients is displayed.

The selection field I13 selectably displays disease identifiers of a plurality of kinds of diseases. In addition, the selection field I13 may also selectably display a disease identifier indicative of a statistical group relating to kinds of diseases. As the statistical group relating to kinds of diseases, use can be made of, as appropriate, the entirety of diseases that can be classified as disease classification information. Besides, the disease identifiers may be displayed in a hierarchical form. For example, as illustrated in FIG. 14, “heart failure” that is an upper layer, and “HFrEF” and “HFpEF” that are lower layers thereof, are displayed. Because of the initial screen 12, a patient graph is not displayed in the display field I15.

If step SC1 is executed, the processing circuitry 51 selects a patient and/or a disease of the display target by implementing the selection function 512 (step SC2). When selecting the patient, the user selects the patient identifier displayed in the selection field I12 through the input interface 53. When selecting the disease, the user selects the disease identifier displayed in the selection field I13 through the input interface 53. Both the patient and the disease may be selected, or only the patient or only the disease may be selected.

Furthermore, in step SC2, by implementing the selection function 512, the processing circuitry 51 selects the medical care event category of the display target. For example, the user can select, through the input interface 53, a desired medical care event category from among medical care event categories displayed in the selection field I14. The number of medical care event categories that are selected may be one or more.

If step SC2 is executed, the processing circuitry 51 generates, by implementing the visualization graph generation function 513, a visualization graph corresponding to the patient and/or disease of the display target and the medical care event category, which are selected in step SC2 (step SC3). For example, when the patient and the disease are selected, the processing circuitry 51 reads the individual situation information from the patient individual situation DB corresponding to the selected target patient, and allocates the individual situation information to the medical knowledge graph. Specifically, the processing circuitry 51 reads the graph feature and the patient feature with respect to each of the nodes, and allocates the graph feature and the patient feature to the node, and, furthermore, the processing circuitry 51 reads the disease weight and disease influence level corresponding to the selected target disease, and allocates the disease weight and disease influence level to the node. As described above, there are the graph feature and patient feature which are based on the patient graph before convolution, and there are the graph feature and patient feature which are based on the patient graph after convolution, and the user can freely set the graph feature and patient feature from among these.

After allocating the individual situation information to each node, the processing circuitry 51 extracts nodes corresponding to the medical care events belonging to the selected medical care event category, and edges connecting the nodes. When one medical care event category is selected, the nodes corresponding to the medical care events belonging to the one medical care event category are extracted. When a plurality of medical care event categories are selected, the nodes corresponding to the medical care events belonging to the medical care event categories are extracted. Next, the processing circuitry 51 extracts a partial patient graph including the extracted nodes and edges, and generates a visualization graph that visualizes the extracted partial patient graph. At this time, the processing circuitry 51 sets the display mode of each node of the visualization graph in accordance with the individual situation information allocated to the node. For example, the display color of the node is set in accordance with the patient feature and graph feature, and the display size of the node is set in accordance with the disease weight and the disease influence level.

Note that the generation procedure of the visualization graph is not limited to the above-described procedure. The partial patient graph corresponding to the medical care events belonging to the selected medical care event category may be first extracted, and then the individual situation information may be allocated to the nodes.

If step SC3 is executed, the processing circuitry 51 displays, by implementing the display control function 514, the visualization graph generated in step SC3 (step SC4). The visualization graph is displayed on the display 55.

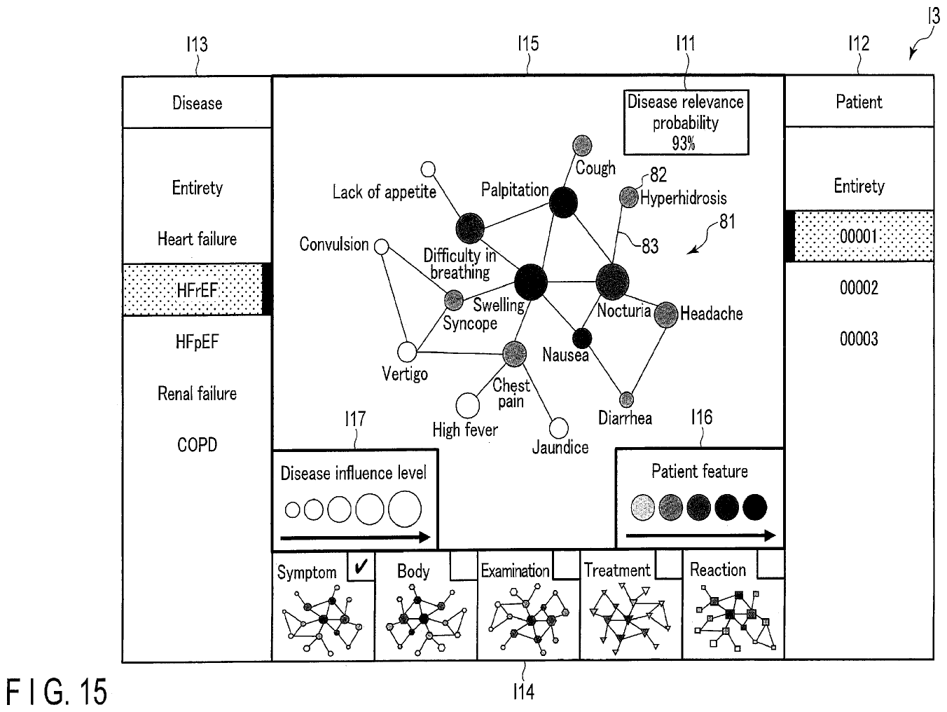

As illustrated in FIG. 15, the display field I15 of the display screen 13 displays the visualization graph generated in step SC3. The nodes of the visualization graph are displayed with display colors and display sizes corresponding to the patient feature and disease influence level of the target patient. The display field I15 may display the display field I11 that indicates the disease relevance probability of the target patient. The disease relevance probability may be read from the patient individual situation DB of the target patient. FIG. 15 illustrates, by way of example, a visualization graph relating to the target patient “00001”, target disease “HFrEF” and medical care event category “symptom”.

As described above, the visualization graph is a display object that visualizes the patient graph which represents a series of medical care events occurring in the target patient by the nodes connected by edges in accordance with a mutual relationship. Each node is emphasized by the patient feature and disease influence level, which are unique to the target patient. By observing the visualization graph, the user can visually recognize what medical care event is involved in the development of the target disease. Moreover, by observing the visualization graph, the user can understand the essential feature of the disease. Besides, the understanding of the mechanism of the disease is promoted.

If step SC4 is executed, the medical knowledge graph display process by the processing circuitry 51 ends.

The above-described medical knowledge graph display process is merely an example, and is not limited to this. For example, in the above-described embodiment, it is assumed that one patient is selected, but the entirety of patients may be selected.

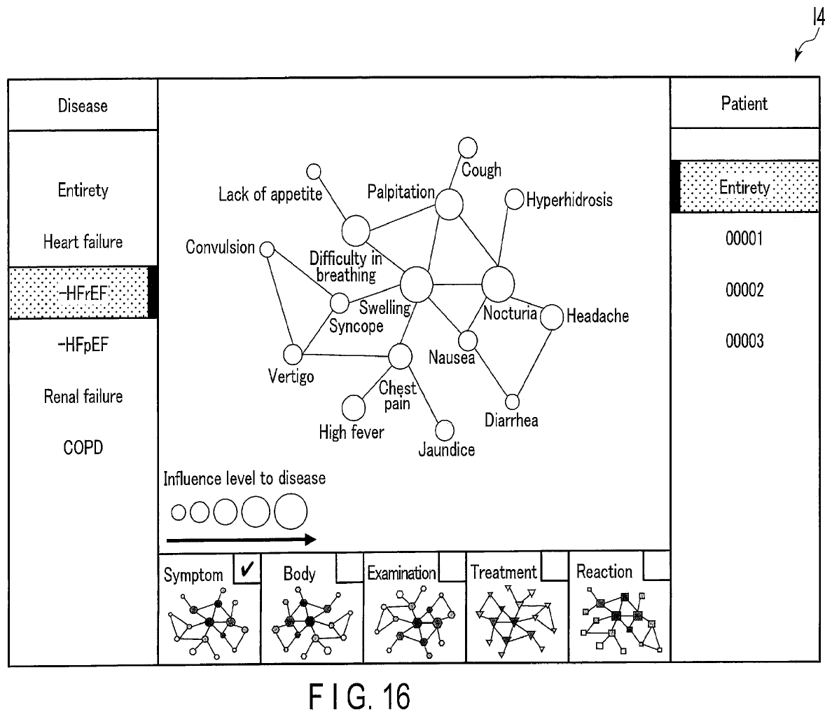

FIG. 16 is a view illustrating a display example of the visualization graph relating to the entirety of patients. As illustrated in FIG. 16, the display field. I11 of a display screen 14 displays a visualization graph that visualizes a patient graph relating to the entirety of patients. An example of the generation procedure of the visualization graph relating to the entirety of patients is as follows.

It is now assumed that the entirety of patients and the specific disease are selected in step SC2. In this case, the processing circuitry 51 reads the patient individual situation DB of all patients, computes statistical values based on the disease influence level of the specific disease relating to all patients with respect to the respective nodes, and allocates the calculated statistical values to the nodes. Then, the processing circuitry 51 displays the nodes in the display mode of the display size or the like corresponding to the allocated statistical values. When the entirety of patients is selected, the patient feature is not allocated to the nodes. Thus, the nodes are displayed in the display mode corresponding to only the disease influence level. In this case, the auxiliary information 116 indicative of the relation between the patient feature and the display color may not be displayed.

Through the visualization graph relating to the entirety of patients, it becomes possible to recognize the disease influence level of the entirety of patients with respect to the respective medical care events. By displaying the visualization graph relating to the entirety of patients and the visualization graph relating to the specific patient alternately or in parallel, it also becomes possible to recognize the specificity or the like of the disease influence level of the specific patient in regard to each medical care event.

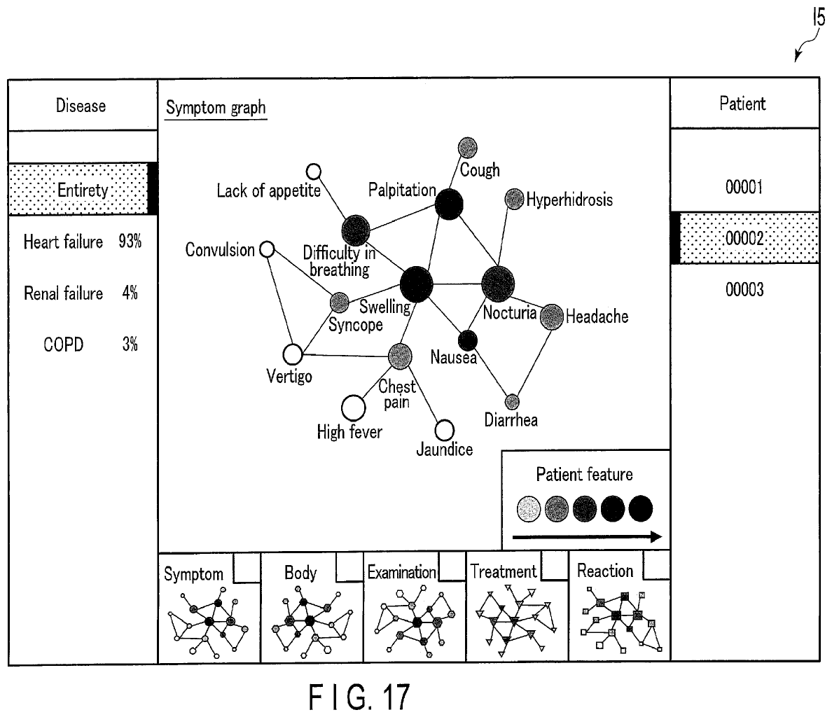

In another example, the entirety of diseases may be selected. FIG. 17 is a view illustrating a display example of the visualization graph relating to the entirety of diseases. As illustrated in FIG. 17, the display field I11 of a display screen 15 displays a visualization graph that visualizes a patient graph relating to the entirety of diseases. An example of the generation procedure of the visualization graph relating to the entirety of diseases is as follows.

It is now assumed that the specific patient and the entirety of diseases are selected in step SC2. In this case, the processing circuitry 51 reads the patient individual situation DB of the specific patient, and allocates the patient feature of the specific patient to the respective nodes. Then, the processing circuitry 51 displays the nodes in the display mode of display colors or the like corresponding to the allocated patient feature. When the entirety of patients is selected, the disease influence level is not allocated to the nodes. Thus, the nodes are displayed in the display mode corresponding to only the patient feature. When the entirety of patients is selected, as illustrated in FIG. 17, it is preferable that the disease relevance probability is also described along with each disease identifier in the selection field I13. For example, “heart failure 93%”, “renal failure 4%”, and “COPD 3%” are displayed. The disease relevance probability may be read from the patient individual situation DB.

Through the visualization graph relating to the entirety of diseases, it becomes possible to recognize the patient feature over the entirety of diseases of the specific patient in regard to individual medical care events. By displaying the visualization graph relating to the entirety of diseases and the visualization graph relating to the specific disease alternately or in parallel, it also becomes possible to recognize the specificity or the like of the patient feature of the specific disease in regard to each medical care event.

As described above, the medical information display apparatus 5 according to the present embodiment includes the processing circuitry 51. The processing circuitry 51 stores the medical knowledge graph including the nodes corresponding to the medical care events and the edges representing the relationship between the nodes. The patient feature and/or disease influence level of each patient is allocated to the nodes of the medical knowledge graph. The processing circuitry 51 specifies the patient and/or disease of the display target. The processing circuitry 51 displays the visualization graph that visualizes the medical knowledge graph in accordance with the patient feature and/or disease influence level relating to the patient and/or disease of the display target.

The visualization graph is a display object that visualizes the patient graph which represents a series of medical care events occurring in the target patient by the nodes connected by edges in accordance with a mutual relationship. Each node is emphasized by the patient feature and disease influence level, which are unique to the target patient. By observing the visualization graph, the user can visually recognize what medical care event is involved in the development of the target disease. Moreover, by observing the visualization graph, the user can understand the essential feature of the disease. Besides, the understanding of the mechanism of the disease is promoted.

### APPLIED EXAMPLES

Hereinafter various applied examples relating to the present embodiment will be described. In the description below, structural elements having substantially the same functions as in the above-described embodiments are denoted by like reference numerals, and an overlapping description is given only where necessary.

### Applied Example 1

In some embodiments described above, it is assumed that the relevance or irrelevance of the medical care event is allocated as the graph feature to each of the nodes. However, the embodiments are not limited to this. As the graph feature, an order of occurrence, a count of occurrences, and/or a degree of occurrence is further allocated to each of the nodes according to Applied Example 1, in addition to the relevance or irrelevance of the medical care event. The order of occurrence means an order of occurrence of the medical care event. The count of occurrences is the count of occurrences of the medical care event. The degree of occurrence is a degree of the medical care event. It is assumed that the order of occurrence, the count of occurrences, and/or the degree of occurrence is included in the medical care event information. Alternatively, the order of occurrence, the count of occurrences, and/or the degree of occurrence may be calculated by analyzing the medical care event information.

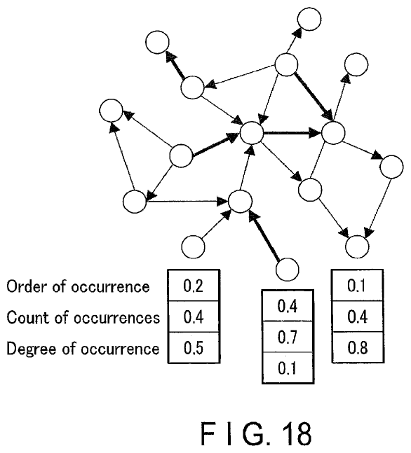

FIG. 18 is a conceptual view of allocation of the order of occurrence, the count of occurrences, and the degree of occurrence. As illustrated in FIG. 18, values indicative of the order of occurrence, the count of occurrences, and the degree of occurrence are allocated as graph features to each of the nodes of the patient graph. In one example, it is preferable that a numerical value indicative of a relative order of occurrence between a plurality of medical care events is allocated as the order of occurrence. In another example, in order to express the order of occurrence and the interval of occurrence by one value, a numerical value (e.g. 0) indicative of the date of the start of a period for cutting out a medical care event, and a numerical value (e.g. 1) indicative of the date of the end of the period, are used as references, and a numerical value may be relatively allocated to a date between these dates. In one example, it is preferable that a numerical value indicative of the count of occurrences, per se, is allocated as the count of occurrences. In another example, in order to taken into account the difference in the count of occurrences between the medical care events, a numerical value of the count of occurrences, per se, may be normalized by a reference (an average value or a maximum value) for each medical care event, and the normalized numerical value may be used as the count of occurrences. As the degree of occurrence, for example, a numerical value indicative of the strength or the like of a symptom is allocated when the medical care event is the symptom; a numerical value indicative of an examination value or the like of an examination is allocated when the medical care event is the examination; a numerical value indicative of a dosage of a drug is allocated when the medical care event is a treatment by the drug; and a numerical value indicative of the strength or the like of a treatment reaction is allocated when the medical care event is the treatment reaction. It is not necessary that all of the order of occurrence, the count of occurrences and the degree of occurrence be allocated, and one kind or two kinds among these may be allocated. Although not illustrated in FIG. 18, it is assumed that a value indicative of the relevance or irrelevance of the medical care event is also allocated to each node.

In Applied Example 1, the graph feature of each node is given as a multidimensional feature composed of the relevance or irrelevance of the medical care event, the order of occurrence, the count of occurrences, and/or the degree of occurrence. By training a machine learning model by using the patient graph in which the multidimensional graph feature is allocated, it becomes possible to generate a trained model that takes into account the order of occurrence of the medical care event, the count of occurrences, and/or the degree of occurrence. By taking into account the order of occurrence of the medical care event, the count of occurrences, and/or the degree of occurrence, it is expected that the estimation accuracy of the medical judgment information, such as the disease relevance probability, is enhanced.

### Applied Example 2

In some embodiments described above, the period of the medical care event information, which is mapped on the medical knowledge graph, is not restricted. The processing circuitry 41 according to Applied Example 2 changes the period of medical care event information to be mapped, in accordance with the kind of the disease of the estimation target. Specifically, a plurality of machine learning models are prepared in accordance with the kind of the disease of the estimation target. It is assumed that the kinds of diseases of estimation targets are, for example, an acute disease and a chronic disease.

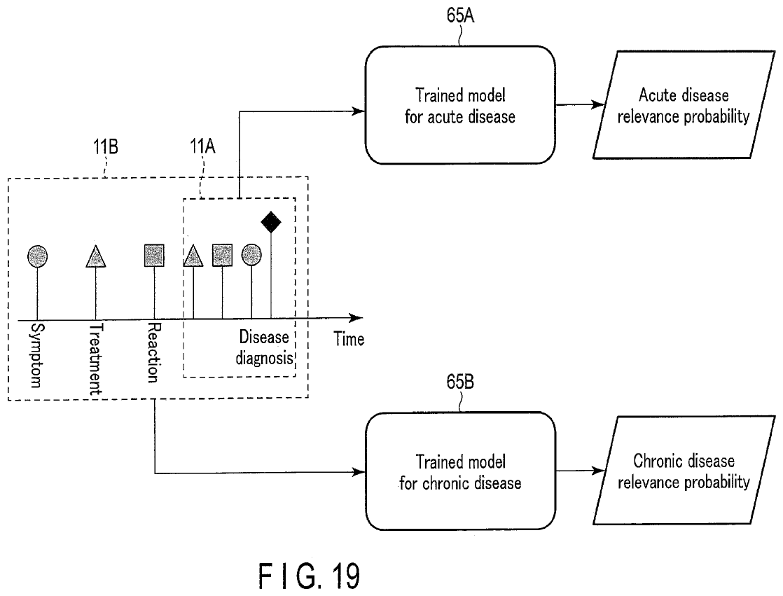

FIG. 19 is a view schematically illustrating inputs and outputs of two machine learning models 65A and 65B according to Applied Example 2. As illustrated in FIG. 19, the machine learning models 65A and 65B according to Applied Example 2 include a machine learning model 65A for an acute disease, and a machine learning model 65B for a chronic disease. A patient map, on which short-period medical care event information 11A is mapped, is input to the machine learning model 65A. The short-period medical care event information 11A includes medical care event information occurring in a relatively short period dating back from disease diagnosis. Note that in FIG. 19, medical care event categories are expressed by shapes of nodes. For example, a circle indicates a symptom category, a triangle indicates a treatment category, and a rectangle indicates a reaction category. The machine learning model 65A is trained on the basis of supervised learning that is based on the patient map and disease information of acute diseases. To be more specific, the learning parameters of the machine learning model 65A are trained based on supervised learning in which the patient map is an input sample and the disease information of acute diseases is a teaching sample. Thereby, a trained model is generated, the trained model inputting therein the patient map on which the short-period medical care event information 11A is mapped, and estimating disease classification information of acute diseases.

Similarly, a patient map, on which long-period medical care event information 11B is mapped, is input to the machine learning model 65B. The long-period medical care event information 11B includes medical care event information occurring in a relatively long period dating back from disease diagnosis. The machine learning model 65B is trained on the basis of supervised learning that is based on the patient map and disease information of chronic diseases. To be more specific, the learning parameters of the machine learning model 65B are trained based on supervised learning in which the patient map is an input sample and the disease information of chronic diseases is a teaching sample. Thereby, a trained model is generated, the trained model inputting therein the patient map on which the long-period medical care event information 11B is mapped, and estimating disease classification information of chronic diseases.

At the time of disease estimation, the processing circuitry 31 determines the period of the medical care event information to be mapped, in accordance with the kind of the disease of the classification target. For example, when the kind of disease is an acute disease, the processing circuitry 31 determines that the period of the medical care event information to be mapped is a short period, and, when the kind of disease is a chronic disease, the processing circuitry 31 determines that the period of the medical care event information to be mapped is a long period. Then, the processing circuitry 31 extracts the medical care event information of the period suited to each trained model, from the history of the medical care event information of the target patient. Specifically, the processing circuitry 31 extracts the medical care event information of the short period from the history of the medical care event information of the target patient, generates the patient map by mapping the medical care event information of the short period on the medical knowledge graph, inputs the patient map to the trained model for acute diseases, and estimates the disease classification information of acute diseases. In addition, the processing circuitry 31 extracts the medical care event information of the long period from the history of the medical care event information of the target patient, generates the patient map by mapping the medical care event information of the long period on the medical knowledge graph, inputs the patient map to the trained model for chronic diseases, and estimates the disease classification information of chronic diseases.

According to Applied Example 2, the length of the period of the medical care event information to be mapped on the medical knowledge graph is made different between the machine learning model for acute diseases and the machine learning model for chronic diseases. The kinds of diseases with different periods of medical care event information to be considered are coped with by generating different trained models. By using these trained models, the estimation accuracy of the disease classification information can be enhanced.

### Applied Example 3

In some embodiments described above, it is assumed that the convolution process by the graph convolution layer does not distinguish the edge relation type. A graph convolution layer according to Applied Example 3 switches parameters, such as the GCN weight, of the convolution process in accordance with the edge relation type of process-target edges connected to process-target nodes. Specifically, the edge relation type is a cause-and-effect direction, a cause-and-effect strength and/or a strength of correlation between medical care events relating to process-target edges. In addition, the edge relation type may be a combination of a category of a medical care event of the process-target node to which the process-target edge is connected, and a category of a medical care event of an adjacent node that neighbors the process-target node. It is assumed that the edge relation type in Applied Example 3 described below is the combination of the categories of the medical care events.

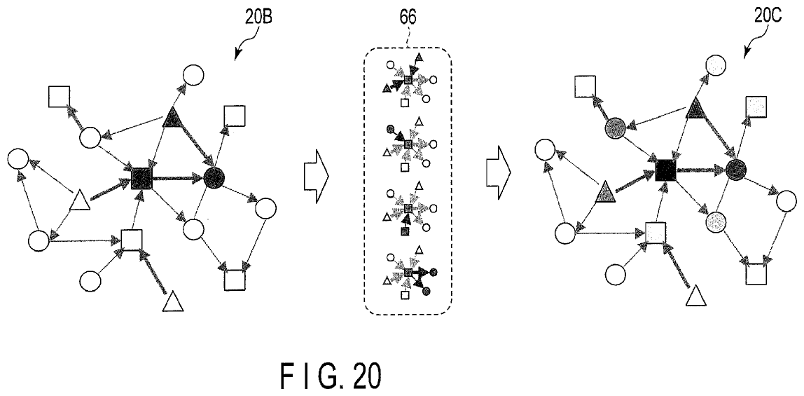

FIG. 20 is a view schematically illustrating a convolution process by a graph convolution layer 66 according to Applied Example 3. As illustrated in FIG. 20, a patient graph 20B includes a plurality of nodes belonging to a plurality of medical care event categories. The graph convolution layer 66 successively switches and executes a plurality of convolution processes corresponding to the medical care event categories. Thereby, a patient graph 20C after convolution is generated. Specifically, the graph convolution layer 66 includes a plurality of filter layers corresponding to the convolution processes. Each filter layer includes parameters such as a GCN weight, which are trained with respect to each of mutually different medical care event categories. For example, a first filter layer is trained with respect to only the nodes belonging to the findings category, and a second filter layer is trained with respect to only the nodes belonging to the symptom category. Arithmetic operation results of the respective filter layers are integrated and output.

The convolution process, in which the edge relation type is taken into account, is described by equation (6) below of R-GCN (Relational Graph Convolutional Network). As indicated in equation (6), a graph feature X′ after convolution of the process-target node is computed by a sum of the first term and the second term. The first term is a product of a graph feature Xⱼ before convolution of an adjacent node j and a weight matrix Wᵣ before convolution of an edge relation type r. To be more specific, the first term is a sum of products of the graph feature Xⱼ, the weight matrix Wᵣ and a normalization constant 1/c_{i,r}, over all combinations of the edge relation type r and adjacent node j. Note that Nᵢ^{r} in equation (6) represents an index of an adjacent node of the edge relation type r of a node i. The second term is computed as a sum of a product of a graph feature Xᵢ before convolution of a process-target node i and a weight matrix W₀ of a self-loop before convolution. As indicated in equation (6), the weight matrix Wᵣ is determined for each edge relation type r, and the product of the graph feature Xⱼ and the weight matrix Wᵣ is computed. Note that the weight matrix Wᵣ and the weight matrix Wₒ are GCN weights of the graph convolution layer, and are examples of learning parameters.

$${X}_{i}^{′} = {\sum }_{r ∈ R} {\sum }_{j ∈ {N}_{i}^{r}} \frac{1}{{c}_{i , r}} {W}_{r} {X}_{j} + {W}_{0} {X}_{i} \qquad ( 6 )$$

As described above, the graph convolution layer including a plurality of filter layers in accordance with edge relation types is designed. Like some embodiments described above, a readout layer and a dense layer are successively connected to a rear stage of the graph convolution layer. The filter layers are provided in a manner to share the rear-stage readout layer and dense layer. In addition, the filter layers may share a part of the graph convolution layer.

Like some embodiments described above, the machine learning model according to Applied Example 3 is trained on the basis of supervised learning that is based on the patient graph and disease information. Thereby, the trained model including filter layers corresponding to edge relation types can be generated. Note that the graph convolution layer according to Applied Example 3 may switch the parameters, such as the GCN weight, of the graph convolution process in accordance with the kind of medical judgment information. As described above, the kinds of medical judgment information are the disease classification information, prognosis estimation information, and severity level classification information. In addition, when the medical judgment information is the disease classification information, the parameters, such as the GCN weight, of the graph convolution layer may be switched in accordance with the kind of disease.

According to Applied Example 3, by dividing the filter layer in accordance with the edge relation type, the learning parameters can be trained in accordance with the edge relation type. Thereby, the accuracy of the graph convolution by the graph convolution layer is enhanced. Furthermore, by extension, the estimation accuracy of the medical judgment information of the disease relevance probability or the like is enhanced.

### Applied Example 4

In some embodiments described above, it is assumed that the disease classification information is estimated as the medical judgment information. However, the medical judgment information is not limited to the disease classification information, and may be prognosis estimation information such as a survival period of a patient, or may be severity level classification information such as a cancer stage classification. Besides, the medical judgment information may be a combination of at least two of the disease classification information, the prognosis estimation information and the severity level classification information.

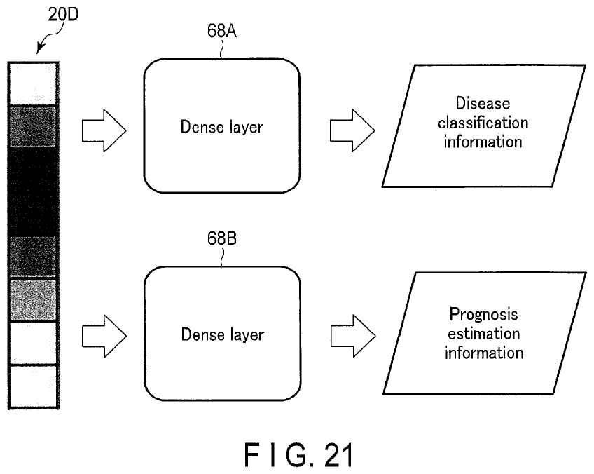

FIG. 21 is a view illustrating an example of inputs and outputs of dense layers 68A and 68B according to Applied Example 4. As illustrated in FIG. 21, a machine learning model according to Applied Example 4 includes two dense layers 68A and 68B. The first dense layer 68A inputs therein a feature vector 20D that is input from a front-stage readout layer (not shown), and outputs disease classification information. The second dense layer 68B inputs therein the same feature vector 20D, and outputs prognosis estimation information.

In the machine learning process, the machine learning model including the graph convolution layer 66, readout layer 67, first dense layer 68A and second dense layer 68B is trained based on multitask learning in which a teaching sample for the first dense layer 68A is disease classification information, a teaching sample for the second dense layer 68B is prognosis estimation information, and a patient graph (not shown) is an input sample. By the multitask learning, the first dense layer 68A inputs therein the feature vector 20D, and the learning parameters such as the disease weight are trained in such a manner as to output the disease classification information, and the second dense layer 68B inputs therein the feature vector 20D, and the learning parameters such as the disease weight are trained in such a manner as to output the prognosis estimation information. Thus, the trained model, which inputs therein the patient graph and outputs the disease classification information and the prognosis estimation information, is generated.

According to Applied Example 4, the machine learning model is trained by the multitask learning based on the patient graph, and two or more of the disease classification information, the prognosis estimation information and the severity level classification information. Thereby, two or more kinds of medical judgment information can be estimated from one patient graph. Thus, the usefulness of the trained model is enhanced.

### Applied Example 5

The individual elements of the above-described Applied Examples 1 to 4 can freely be combined. A machine learning model according to Applied Example 5 is constructed by combining the elements of Applied Examples 1 to 4.

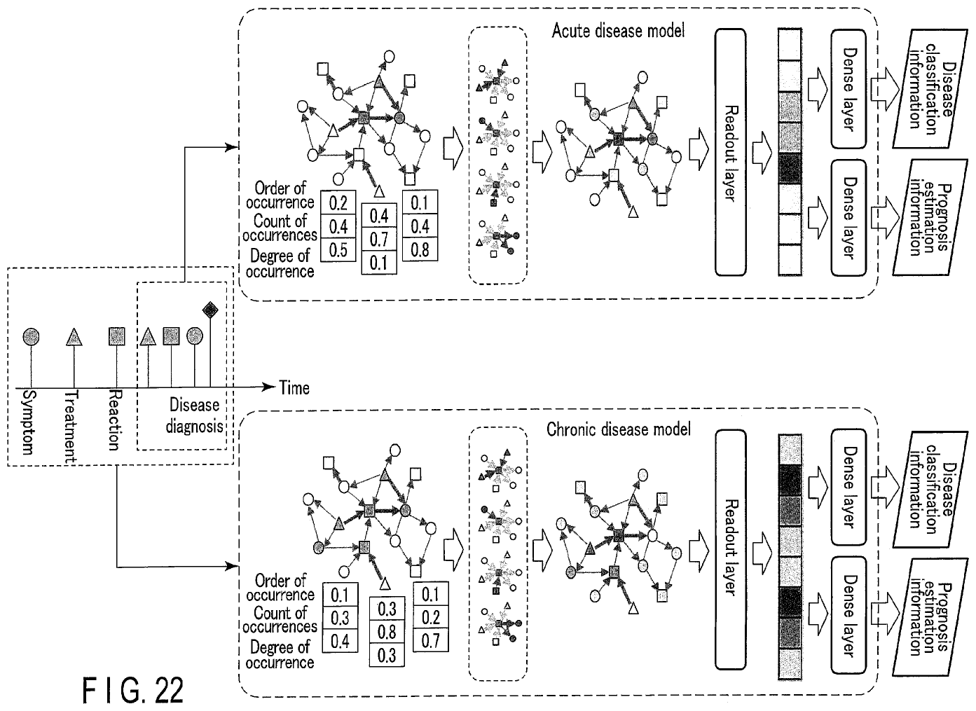

FIG. 22 is a view schematically illustrating machine learning models according to Applied Example 5. As illustrated in FIG. 22, a trained model includes a machine learning model for acute diseases, and a machine learning model for chronic diseases. The machine learning models are different with respect to the period of the medical care event information mapped on the input patient graph. A patient graph, on which the medical care event information of a short period is mapped, is input to the machine learning model for acute diseases. A patient graph, on which the medical care event information of a long period is mapped, is input to the machine learning model for chronic diseases. In the patient graph, the order of occurrence of the medical care event, the count of occurrences, and the degree of occurrence are allocated, as well as the relevance or irrelevance of the disease. The graph convolution layer of each machine learning model includes different filter layers in accordance with edge relation types. Each model includes two dense layers. The first dense layer inputs therein a feature vector and outputs disease classification information, and the second dense layer inputs therein the feature vector and outputs prognosis estimation information.

The machine learning model according to Applied Example 5 is trained based on multitask learning in which a teaching sample for the first dense layer is disease classification information, and a teaching sample for the second dense layer is prognosis estimation information. Thereby, the trained model can be generated which executes graph convolution according to the edge relation type with respect to the acute disease and the chronic disease, and outputs the disease classification information and the prognosis estimation information.

### Applied Example 6

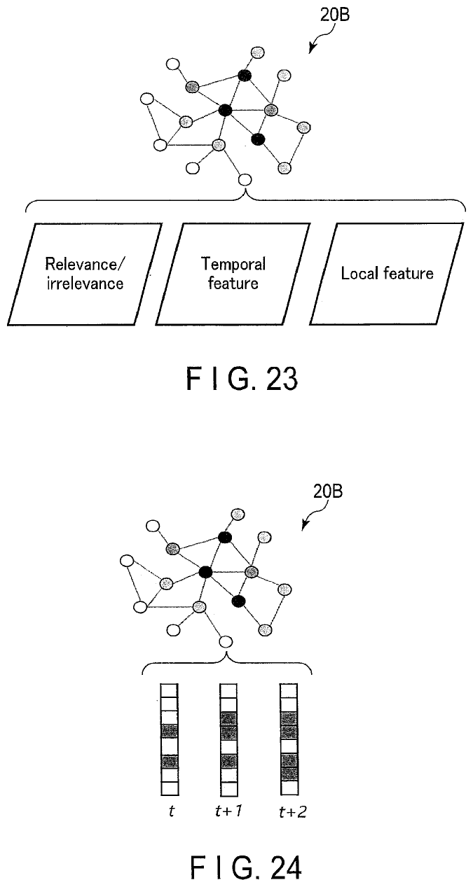

FIG. 23 is a view representing an outline of graph features according to Applied Example 6. As illustrated in FIG. 23, as the graph feature, medical care event information, such as a relevance/irrelevance feature, a temporal feature and/or a local feature, is allocated to each node of the patient graph 20B. The relevance/irrelevance feature is information indicative of a degree of relevance or a degree of irrelevance to the medical care event corresponding to the node. The temporal feature is information relating to a time of occurrence of the medical care event. Specifically, the temporal feature is the date/time of occurrence of the medical care event, the order of occurrence, the count of occurrences, or the like. The local feature is information relating to a location of occurrence of the medical care event. Specifically, the local information is a position of occurrence of the medical care event, a location of occurrence, or the like. The position of occurrence is sensor information of GPS (Global Positioning System) or the like, which represents a point of occurrence of the medical care event. The location of occurrence is an address or a name of a medical institution, a hospital department, the home, or a hospital, where the medical care event is diagnosed.

Hereinafter, it is assumed that the graph feature according to Applied Example 6 includes the relevance/irrelevance feature, temporal feature and local feature.

By implementing the mapping function 312, the processing circuitry 31 according to Applied Example 6 generates a patient graph 20B of a target patient by mapping the medical care event information on the medical knowledge graph. Here, the medical care event information includes the relevance/irrelevance feature, temporal feature and local feature in regard to each medical care event. Thereby, the patient graph 20B is generated in which the relevance/irrelevance feature, temporal feature and local feature are allocated to the nodes.

Thereafter, by implementing the estimation function 313, the processing circuitry 31 applies to the trained model 60 the patient graph 20B including the nodes to which the relevance/irrelevance feature, temporal feature and local feature are allocated, and estimates medical judgment information such as the disease classification information 71. According to Applied Example 6, the medical judgment information can be estimated by taking into account the relevance/irrelevance feature, temporal feature and local feature of each medical care event. Note that it is not necessary that all of the relevance/irrelevance feature, temporal feature and local feature be allocated to the nodes, but only two or one among these may be allocated.

### Applied Example 7

FIG. 24 is a view representing an outline of a graph feature according to Applied Example 7. As illustrated in FIG. 24, as the graph feature, time-series medical care event information, which is composed of a plurality of pieces of medical care event information with different time instants of occurrence, is allocated to each node of the patient graph 20B. The value of the medical care event information varies with time. For example, the relevance/irrelevance of the medical care event varies with time. Time-of-occurrence information is allocated as meta-information to the medical care event information. The time-of-occurrence information is a time stamp of the medical care event information, and, for example, the date/time of diagnosis or the date/time of record of the medical care event is allocated to the time-of-occurrence information. For example, as illustrated in FIG. 24, medical care event information at time t, medical care event information at time t+1, and medical care event information at time t+2 are allocated to each node as the graph feature.

The processing circuitry 31 according to Applied Example 7 estimates the medical judgment information, based on the time-series graph feature.

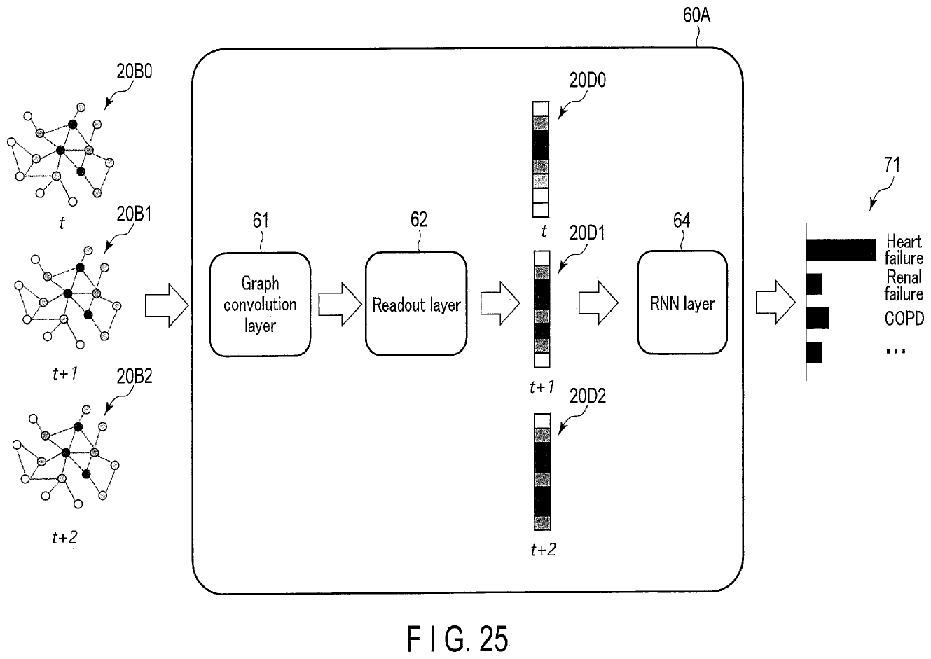

FIG. 25 is a view illustrating an example of an estimation process of medical judgment information according to Applied Example 7. As illustrated in FIG. 25, a trained model 60A according to Applied Example 7 includes a graph convolution layer 61, a readout layer 62 and an RNN (Recurrent Neural Network) layer 64. The RNN layer 64 outputs disease classification information 71 from time-series graph feature 20D.

As illustrated in FIG. 25, it is assumed that process-targets are a patient graph 20B0 to which the graph feature of time t is allocated, a patient graph 20B1 to which the graph feature of time t+1 is allocated, and a patient graph 20B2 to which the graph feature of time t+2 is allocated. The processing circuitry 31 inputs the patient graph 20B0, patient graph 20B1 and patient graph 20B2 to the graph convolution layer 61, outputs a patient graph after convolution corresponding to the patient graph 20B0, a patient graph after convolution corresponding to the patient graph 20B1 and a patient graph after convolution corresponding to the patient graph 20B2, inputs the patient graphs after convolution to the readout layer 62, and outputs a feature vector 20D0 of time t, a feature vector 20D1 of time t+1 and a feature vector 20D2 of time t+2. The feature vector 20D0 is a vector expression of the graph feature of time t, the feature vector 20D1 is a vector expression of the graph feature of time t+1, and the feature vector 20D2 is a vector expression of the graph feature of time t+2.

As illustrated in FIG. 25, the processing circuitry 31 inputs the feature vector 20D0, feature vector 20D1 and feature vector 20D2 to the RNN layer 64, and outputs a single piece of disease classification information 71. By utilizing the RNN layer 64, it becomes possible to obtain the relevance probability of various diseases by utilizing the medical care event information of a plurality of serial time instants.

### Applied Example 8

In Applied Example 7, it is assumed that the time-series graph feature is allocated to each node. In Applied Example 8, the patient graph itself varies in a time-series manner.

Processing circuitry 41 of a medical information learning apparatus 4 according to Applied Example 8 continues to obtain training samples (medical care information) with different time instants of occurrence, even after the trained model is once generated. As described above, the training sample is the combination of the medical care event information and the disease information. Based on the obtained pieces of medical care information, the processing circuitry 41 executes continuous learning for learning parameters of the machine learning model regularly or irregularly. By the continuous learning, time-series trained models with different time instants of occurrence are generated. Note that the learning parameters include an adjacency matrix and/or GCN weights. Patient graphs before convolution at respective time instants are applied to the trained models at respective time instants, and patient graphs after convolution at respective time instants are generated. Thereby, time-series patient graphs, in which the graph feature of each node and the connection relation between the nodes vary in a time-series manner, can be generated.

The trained models at respective time instants of occurrence, which constitute the time-series trained models, are trained according to the processing procedure illustrated in FIG. 10, based on the training samples obtained up to the time instant of occurrence. At this time, in order to suppress a great change of characteristics of the patient graph before and after the update of the adjacency matrix A and/or GCN weights W, a regularization term may be provided in a loss function, as indicated in equations (7) and (8) below. Equation (7) is a regularization term L_{reg} relating to the update of the adjacency matrix A, and is expressed by Kullback-Leibler information between an adjacency matrix A at a process reference time instant and an adjacency matrix A′ at another time instant. Equation (8) is a regularization term L_{reg} relating to the update of the GCN weight W^{(l)}, and is expressed by Kullback-Leibler information between a GCN weight W^{(l)} at a process reference time instant and a GCN weight W′^{(l)} at another time instant.

L_{reg}=D_{KL}(A∥A′) (7)

L_{reg}=D_{KL}(W^{(l)}∥W′^{(l)}) (8)

### Applied Example 9

In some embodiments described above, it is assumed that spatial information such as the local feature is allocated as the graph feature to each node of the patient graph. In Applied Example 9, a concept of space is added to the patient graph itself.

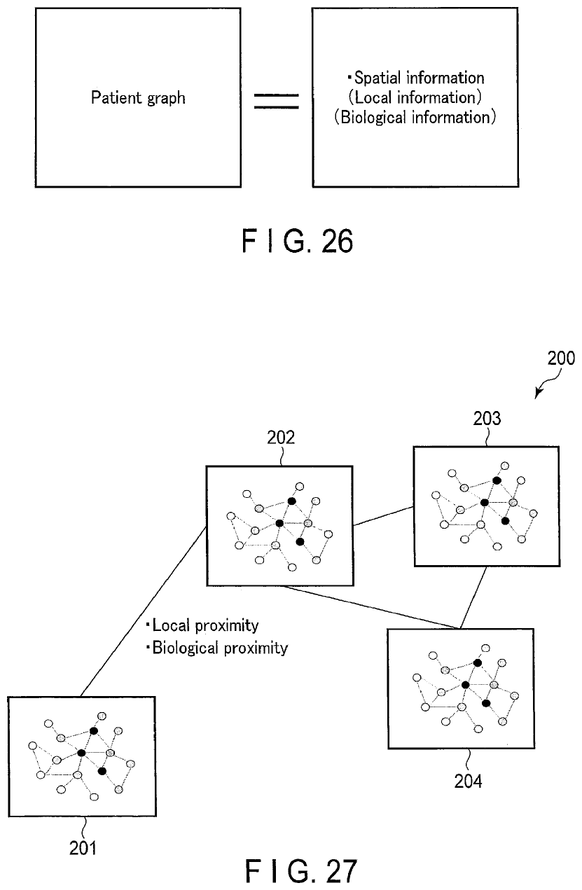

FIG. 26 is a view schematically illustrating a relationship between a patient graph and spatial information according to Applied Example 9. As illustrated in FIG. 26, spatial information relating to the target patient of each patient graph is allocated to the patient graph. The spatial information includes local information and biological information as elements of the spatial information. The local information may be sensor information of a GPS or the like which indicates the present position of the target patient, or may be the position of a medical institution where the target patient undergoes a medical examination, the address of the home of the target patient, or the position of a hospital room or the like where the target patient is hospitalized. The biological information includes the blood relationship, the medical history, and the gene arrangement of the target patient. By embedding the spatial information in the patient graph, it is possible to construct a network (hereinafter “patient graph network”) in which a plurality of patient graphs are arranged in accordance with the presence/absence or the degree of the relation of the spatial information.

FIG. 27 is a view illustrating a concept of a patient graph network 200. As illustrated in FIG. 27, the patient graph network 200 is composed of a plurality of patient graphs that are connected in accordance with the presence/absence or the degree of the relation of the spatial information. FIG. 27 exemplarily illustrates four patient graphs 201, 202, 203 and 204, but the number of patient graphs is not specifically limited if the number is two or more.

A connection by an edge between patient graphs (for example, patient graph 201 and patient graph 202) means that these patient graphs have a relationship of spatial information. Conversely, non-connection by an edge between patient graphs (for example, patient graph 201 and patient graph 204) means that these patient graphs have no relationship of spatial information. In addition, the distance between patient graphs connected by an edge represents the degree of a relationship of spatial information. As regards the local information, the processing circuitry 41 can evaluate the degree of the relationship of the spatial information, for example, based on the distance between the addresses of the homes or the hospital rooms of both patients. As regards the biological information, the processing circuitry 41 can perform the evaluation, based on the degree of consanguinity of both patients, the medical relationship of the medical histories, and the ratio of coincidence of gene arrangements. The processing circuitry 41 can determine the presence/absence of the relationship of the spatial information, based on the comparison between the degree evaluated by the above method and a threshold. The edge may be formed based on the comprehensive evaluation of the above-described elements of the spatial information, or may be formed in regard to each of the above-described elements.

By allocating the spatial information to the patient graph, it is possible to easily search a patient graph whose spatial information is close to the patient graph of the target patient. In one example, the processing circuitry 41 can extract a patient graph, which is connected to the patient graph of the target patient by an edge, as the patient graph of a patient whose spatial information is close to the target patient.

Note that the spatial information is not limited to the spatial information including both the local information and the biological information, and may be spatial information including only one of the local information and the biological information.

### Applied Example 10

In Applied Example 10, the spatial information according to Applied Example 9 is utilized. Processing circuitry 31 of a medical information processing apparatus 3 according to Applied Example 10 convolutes a graph feature of a patient graph of a patient, who is different from the target patient, into the patient graph of the target patient.

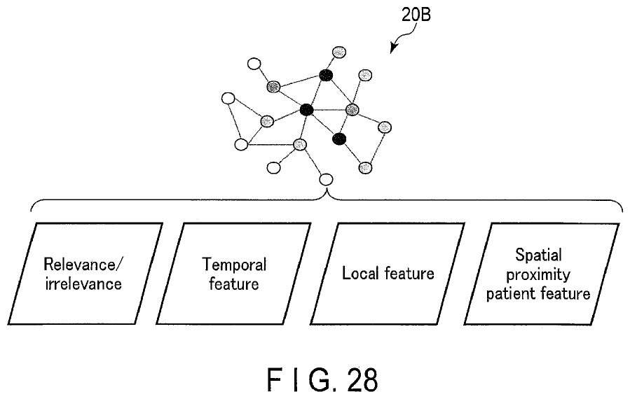

FIG. 28 is a view representing an outline of graph features according to Applied Example 10. As illustrated in FIG. 28, as the graph feature, medical care event information, such as a relevance/irrelevance feature, a temporal feature, a local feature and/or a spatial proximity patient feature, is allocated to each node of the patient graph 20B. The relevance/irrelevance feature, temporal feature and local feature are as described in Applied Example 6. The spatial proximity patient feature is medical care event information of another patient, whose spatial information is close to the target patient. For example, as well as the relevance/irrelevance feature, temporal feature and local feature of the target patient, the relevance/irrelevance feature, temporal feature and local feature of the father and/or mother of the target patient are allocated to the node of the medical care event “headache”.

By implementing the mapping function 312, the processing circuitry 31 according to Applied Example 10 generates a patient graph 20B of the target patient by mapping the medical care event information according to Applied Example 10 on the medical knowledge graph. The medical care event information according to Applied Example 10 includes the relevance/irrelevance feature, temporal feature, local feature and spatial proximity patient feature in regard to each medical care event. The relevance/irrelevance feature, temporal feature and local feature are the medical care event information of the target patient. The spatial proximity patient feature is the medical care event information of another patient, whose spatial information is close to the target patient. Specifically, by implementing the mapping function 312, the processing circuitry 31 according to Applied Example 10 maps the medical care event information of the target patient and the medical care event information of the another patient on the nodes as node features of the nodes. By implementing the mapping function 312, the patient graph 20B is generated in which the relevance/irrelevance feature, temporal feature, local feature and spatial proximity patient feature are allocated to the nodes.

Thereafter, by implementing the estimation function 313, the processing circuitry 31 estimates the medical judgment information such as the disease classification information 71, by applying to the trained model 60 the patient graph 20B including the nodes to which the relevance/irrelevance feature, temporal feature, local feature and spatial proximity patient feature are allocated. According to Applied Example 10, the medical judgment information can be estimated by taking into account the relevance/irrelevance feature, temporal feature, local feature and spatial proximity patient feature of each medical care event. Thereby, the medical judgment information can be estimated in which the medical care information (graph feature) of not only the target patient but also the patient whose spatial information is close to the target patient is taken into account. Note that it is not necessary that all of the relevance/irrelevance feature, temporal feature, local feature and spatial proximity patient feature be allocated to the nodes, but only two or one among these may be allocated.

### Applied Example 11

In some embodiments described above, as regards the display of the visualization graph based on the patient graph, it is assumed that the processing circuitry 31 displays the visualization graph based on a partial patient graph including only the nodes and edges belonging to a part of all of the categories including the symptom, physical finding, examination finding, treatment, treatment reaction, and side effect. However, as illustrated in FIG. 2, the processing circuitry 31 may display the visualization graph based on the entirety of the patient graph over all medical care event categories. At this time, the processing circuitry 31 may display the visualization graph in which the respective categories can be distinguished by visual effects such as colors. Thereby, the entirety of the patient graph can be overlooked.

### Other Embodiments

Some embodiments can be added to, or substituted for, the above embodiments. For example, transfer learning may be executed for the machine learning model. At first, learning parameters of a machine learning model, which estimates disease classification information of a first disease (e.g. acute disease), may be trained, and then some of the learning parameters may be set in a machine learning model that estimates disease classification information of a second disease (e.g. chronic disease) and the remaining learning parameters may be trained. For example, this is useful when the number of training samples of the second disease is small. Note that, as the second disease that is a transfer destination of the transfer learning, a disease having some medical commonness or similarity to the first disease, which is a transfer source of the transfer learning, is appropriate.

Regularization may be taken into account in the machine learning process. This is achieved by adding a freely selected regularization term to a loss function. As the regularization term to be added, an appropriate term is a penalty term that applies a penalty to the magnitude of the norm of the learning parameter, or a penalty term that applies a penalty by an index of independency of a network parameter between diseases.

In the above-described machine learning process, it is assumed that the adjacency matrix that defines the relationship between the edges is not a learning parameter. However, the embodiments are not limited to this, and the adjacency matrix may be trained as a learning parameter in the machine learning process.

The above-described machine learning model may be provided with an additional module such as an attention (Attention) mechanism. For example, an attention mechanism is provided in parallel between the output layer of the readout layer and the input layer of the dense layer. The attention mechanism outputs an attention mask indicative of the degree of emphasis of each node. The dense layer estimates medical judgment information, based on the attention mask and the feature vector. By the provision of the attention mechanism, the feature part of the patient graph, which is useful in estimation of the medical judgment information, is emphasized, and therefore the estimation accuracy of the medical judgment information is enhanced.

In the above-described embodiments, it is assumed that the graph convolution layer, such as the GCN or R-GCN, is used as the model that processes the patient graph. However, the embodiments are not limited to this. As the model that processes the patient graph, use may be made of a Boltzmann machine using a Markov random field (MRF) or conditional random field (CRF), or an application thereof.

It is assumed that the teaching sample is disease information obtained from the medical care information, or disease information obtained from the medical ontology. However, the teaching sample may include both of the disease information obtained from the medical care information and the disease information obtained from the medical ontology. In this case, the machine learning model may output a multi-label classification that outputs the disease information obtained from the medical care information and the disease information obtained from the medical ontology.

According to at least one of the above-described embodiments, the accuracy of medical judgment information can be enhanced.

The term “processor” used in the above description means a CPU, a GPU, or circuitry such as an application specific integrated circuit (ASIC) and programmable logic devices (e.g. a simple programmable logic device (SPLD), a complex programmable logic device (CPLD) and a field programmable gate array (FPGA)). The processor implements a function by reading and executing a program stored in storage circuitry. Note that, instead of storing the program in the storage circuitry, a configuration may be adopted in which the program is directly embedded in the circuitry of the processor. In this case, the processor implements the function by reading and executing the program embedded in the circuitry of the processor. Furthermore, instead of executing the program, the function corresponding to the program may be implemented by a combination of logic circuits. Note that each of the processors of the embodiments may not be constituted as single circuitry for each processor, but a plurality of independent circuits may be combined to constitute one processor and may implement the function of the processor. Besides, the structural elements in FIG. 1, FIG. 3, FIG. 9 and FIG. 12 may be integrated into one processor, and the processor may implement the functions of the structural elements.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing apparatus comprising processing circuitry configured to:
obtain medical care information relating to medical care events of a target patient;
map the medical care information on a first graph to generate a second graph relating to the target patient, the first graph including nodes corresponding to the medical care events and edges indicative of a relationship between the nodes; and
estimate medical judgment information relating to the target patient, based on the second graph relating to the target patient.

2. The medical information processing apparatus of claim 1, wherein the medical care events include an event belonging to at least one category among a symptom, a physical finding, an examination finding, a treatment, a treatment reaction, and a side effect.

3. The medical information processing apparatus of claim 1, wherein
the processing circuitry estimates, by utilizing a trained model, the medical judgment information relating to the target patient, based on the second graph relating to the target patient, and
the trained model is a machine learning model trained such that the machine learning model inputs therein the second graph and outputs the medical judgment information.

4. The medical information processing apparatus of claim 3, wherein the trained model includes:
a graph convolution layer configured to apply a convolution process to the second graph, and configured to output a third graph;
a readout layer configured to convert the third graph to a feature vector; and
a dense layer configured to convert the feature vector to the medical judgment information.

5. The medical information processing apparatus of claim 4, wherein
the graph convolution layer computes, with respect to each of nodes included in the second graph, a feature after a convolution process, based on a feature before the convolution process in regard to a process-target node and an adjacent node to the process-target node, an adjacency matrix indicative of the edge connecting the process-target node and the adjacent node, and a weight on the edge, and
the readout layer converts the feature after the convolution process in regard to each of the nodes to the feature vector.

6. The medical information processing apparatus of claim 4, wherein the processing circuitry displays, on a display device, a visualization graph that visualizes the second graph or the third graph relating to the target patient.

7. The medical information processing apparatus of claim 6, wherein the processing circuitry displays nodes included in the visualization graph in a display mode corresponding to patient features allocated to the nodes included in the second graph or the third graph.

8. The medical information processing apparatus of claim 6, wherein the processing circuitry displays the nodes in a display mode corresponding to disease influence levels that correspond to the nodes and are allocated to the nodes included in the second graph or the third graph.

9. The medical information processing apparatus of claim 6, wherein the processing circuitry extracts, from the second graph or the third graph, a partial graph including nodes of display targets and edges connecting the nodes of the display targets, and displays a visualization graph that visualizes the partial graph.

10. The medical information processing apparatus of claim 6, wherein
the nodes are classified into at least two categories among a symptom, a physical finding, an examination finding, a treatment, a treatment reaction, and a side effect, and
the processing circuitry extracts, from the second graph or the third graph, a partial graph including nodes belonging to categories of display targets and edges connecting the nodes, and displays a visualization graph that visualizes the partial graph.

11. The medical information processing apparatus of claim 6, wherein the processing circuitry adds, to the nodes included in the visualization graph, names or symbols of the medical care events corresponding to the nodes.

12. The medical information processing apparatus of claim 4, wherein
the graph convolution layer switches parameters of the convolution process in accordance with an edge relation type of process-target edges connected to process-target nodes, and
the edge relation type is a cause-and-effect direction, a cause-and-effect strength and/or a strength of correlation between medical care events relating to the process-target edges.

13. The medical information processing apparatus of claim 4, wherein
the graph convolution layer switches parameters of the convolution process in accordance with an edge relation type of process-target edges connected to process-target nodes, and
the edge relation type is a combination of a category of a medical care event of the process-target node to which the process-target edge is connected, and a category of a medical care event of an adjacent node that neighbors the process-target node.

14. The medical information processing apparatus of claim 4, wherein the graph convolution layer switches parameters of the convolution process in accordance with a kind of the medical judgment information.

15. The medical information processing apparatus of claim 4, wherein the graph convolution layer switches parameters of the convolution process in accordance with a kind of a disease in the medical judgment information.

16. The medical information processing apparatus of claim 1, wherein the processing circuitry determines a period of the medical care information to be mapped, in accordance with a kind of a disease of a classification target that is the medical judgment information.

17. The medical information processing apparatus of claim 1, wherein
the medical care information includes an order of occurrence of the medical care event, a count of occurrences of the medical care event, and/or a degree of occurrence of the medical care event, and
the processing circuitry maps the medical care information on the nodes as a node feature.

18. The medical information processing apparatus of claim 1, wherein
the medical care information includes local information and/or temporal information relating to the medical care events,
the processing circuitry maps the local information and/or the temporal information on the nodes as a node feature,
the local information is information relating to a position of occurrence of the medical care event, and
the temporal information is information relating to a time of occurrence of the medical care event.

19. The medical information processing apparatus of claim 18, wherein
a plurality of pieces of medical care information with different time instants of occurrence are allocated to the nodes, and
the processing circuitry estimates, by utilizing a trained model including a graph convolution layer and a recurrent neural network layer, the medical judgment information relating to the target patient from the second graph including the nodes to which the plurality of pieces of medical care information are allocated.

20. The medical information processing apparatus of claim 1, wherein
the medical care information includes medical care information of the target patient, and medical care information of another patient whose spatial information is close to the target patient,
the spatial information includes local information and/or biological information of the another patient, and
the processing circuitry maps the medical care information of the target patient and the medical care information of the another patient on the nodes as node features.

21. The medical information processing apparatus of claim 1, wherein the processing circuitry estimates, as the medical judgment information, at least one information among disease classification information, prognosis estimation information and severity level classification information corresponding to the second graph relating to the target patient.

22. The medical information processing apparatus of claim 1, further comprising a display controller configured to display the second graph relating to the target patient, wherein
the medical care events include an event belonging to at least one category among a symptom, a physical finding, an examination finding, a treatment, a treatment reaction, and a side effect, and
the processing circuitry displays the second graph such that the category is distinguishable.

23. The medical information processing apparatus of claim 1, wherein the first graph is a graph generated based on medical care information of a plurality of patients, or medical ontology.

24. A medical information learning apparatus comprising processing circuitry configured to:
obtain a first graph including nodes corresponding to medical care events, and edges indicative of a relationship between the nodes, and medical care information relating to the medical care events;
generate a second graph in which the medical care information is mapped on the first graph; and
train, based on the second graph and medical judgment information corresponding to the medical care information, a model for estimating the medical judgment information from the second graph.

25. The medical information learning apparatus of claim 24, wherein the processing circuitry obtains the medical judgment information for use as a teaching sample in training of the model.

26. The medical information learning apparatus of claim 25, wherein the medical judgment information is disease information relating to two or more diseases according to a hierarchical structure in nosology.

27. The medical information learning apparatus of claim 25, wherein the processing circuitry specifies the medical judgment information, based on at least one of the medical care information and medical ontology.

28. The medical information learning apparatus of claim 24, wherein the model includes:
a graph convolution layer configured to apply a convolution process to the second graph, and configured to output a third graph;
a readout layer configured to convert the third graph to a feature vector; and
a dense layer configured to convert the feature vector to the medical judgment information.

29. The medical information learning apparatus of claim 24, wherein
the medical judgment information includes at least two pieces of information among disease classification information, prognosis estimation information, and severity level classification information, and
the processing circuitry trains the model by multitask learning based on the second graph and the at least two pieces of information.

30. The medical information learning apparatus of claim 24, wherein the processing circuitry trains the model by transfer learning.

31. The medical information learning apparatus of claim 24, wherein the processing circuitry executes continuous learning for the model, based on a plurality of pieces of the medical care information with different time instants of occurrence.

32. A medical information display apparatus comprising:
a storage device that is a unit configured to store a graph including nodes corresponding to medical care events and edges indicative of a relationship between the nodes, a patient feature and/or a disease influence level being allocated to the nodes; and
processing circuitry configured to select a patient and/or a disease which is a display target, and configured to display, on a display device, a visualization graph that visualizes the graph in accordance with the patient feature and/or the disease influence level relating to the patient and/or the disease which is the display target.

33. The medical information display apparatus of claim 32, wherein when a specific patient is selected as the display target, the processing circuitry displays nodes included in the visualization graph in a display mode corresponding to a patient feature of the specific patient, the patient feature corresponding to the nodes.

34. The medical information display apparatus of claim 32, wherein when a specific disease is selected as the display target, the processing circuitry displays nodes included in the visualization graph in a display mode corresponding to a disease influence level of the specific disease, the disease influence level corresponding to the nodes.

35. The medical information display apparatus of claim 32, wherein when a combination of a specific patient and a specific disease is specified as the display target, the processing circuitry displays nodes included in the visualization graph in a display mode corresponding to a patient feature of the combination, which corresponds to the nodes, and in a second display mode corresponding to a disease influence level of the combination.

36. The medical information display apparatus of claim 32, wherein
the nodes are classified into at least two categories among a symptom, a physical finding, an examination finding, a treatment, a treatment reaction, and a side effect, and
the processing circuitry extracts a partial graph including nodes belonging to a category of a display target, among nodes included in the graph, and edges connecting the nodes, and displays a visualization graph that visualizes the partial graph.

37. The medical information display apparatus of claim 36, wherein the processing circuitry displays a display screen including a display area of the visualization graph, and a selection area of the category of the display target.

38. The medical information display apparatus of claim 32, wherein the processing circuitry displays a display screen including a display area of the visualization graph, and a selection area of the patient and/or the disease of the display target.

39. A medical information processing method comprising:
obtaining medical care information relating to medical care events of a target patient;
mapping the medical care information on a first graph to generate a second graph relating to the target patient, the first graph including nodes corresponding to the medical care events and edges indicative of a relationship between the nodes; and
estimating medical judgment information relating to the target patient, based on the second graph relating to the target patient.
